(19) United States
(12) Patent Application Publication
(10) Pub. No.: US 2025/0107687 A1
(43) Pub. Date: Apr. 3, 2025

(54) FIELD-OF-VIEW CONTROL APPARATUS, FIELD-OF-VIEW CONTROL SYSTEM, FIELD-OF-VIEW CONTROL METHOD, AND RECORDIMG MEDIUM

(71) Applicant: OLYMPUS CORPORATION, Tokyo (JP)

(72) Inventors: Takeo UCHIDA, Tokyo (JP); Masafumi HARAGUCHI, Tokyo (JP)

(73) Assignee: OLYMPUS CORPORATION, Tokyo (JP)

(21) Appl. No.: 18/896,186

(22) Filed: Sep. 25, 2024

Related U.S. Application Data
(60) Provisional application No. 63/542,139, filed on Oct. 3, 2023.

(51) Int. Cl. A61B 1/00; H04N 23/50; H04N 23/60; H04N 23/611; H04N 23/695

(52) U.S. Cl. CPC A61B 1/00006; A61B 1/00009; A61B 1/0002; A61B 1/00149; A61B 1/00179; H04N 23/555; H04N 23/64; H04N 23/695; H04N 23/611

(57) ABSTRACT

A field-of-view control apparatus includes a processor configured to execute: determining a procedural scene; presenting a user with information related to library data for realizing a field of view associated with the procedural scene as an execution candidate for field-of-view adjustment; outputting, if the user wishes to execute the field-of-view adjustment based on the presented library data, an instruction to change a position, an attitude, or a field-of-view direction of an electric scope based on the library data; and presenting, if the user does not wish to execute the field-of-view adjustment based on the presented library data, the user with information related to other library data classified in a same procedural scene as the library data being the execution candidate as an other of the execution candidate. The library data includes at least one relative parameter related to relative positions and attitudes of the electric scope and an observation object.

### CROSS-REFERENCE TO RELATED APPLICATIONS

This application claims the benefit of U.S. Provisional Application No. 63/542,139, filed Oct. 3, 2023, which is hereby incorporated by reference herein in its entirety.

### TECHNICAL FIELD

The present disclosure relates to a field-of-view control apparatus, a field-of-view control system, a field-of-view control method, and a recording medium.

### BACKGROUND

Conventionally, a technique for providing an endoscopic field of view in accordance with a designation by a user during an endoscopic procedure is known (for example, refer to PTL 1). With the technique described in PTL 1, when the user selects an object such as a blood vessel, a nerve, or a tumor to be tracked, an endoscopic field of view is generated in accordance with the selected object to be tracked based on follow parameters such as a distance and a speed set in advance. In the technique described in PTL 1, a follow parameter that corresponds one-to-one is set for each object to be tracked.

### SUMMARY

A first aspect of the present disclosure is a field-of-view control apparatus comprising: one or more processors comprising hardware, the one or more processors being configured to: scene determination processing of determining a present procedural scene based on at least one of an endoscopic image of an observation object acquired by an endoscope, a position and an attitude of the endoscope, a field-of-view direction of the endoscope, or input information of the procedural scene by a user; candidate presentation processing of presenting the user with information related to library data for realizing an endoscopic field of view associated with the determined present procedural scene as an execution candidate for endoscopic field-of-view adjustment; field-of-view adjustment processing of outputting, in a case where the user wishes to execute the endoscopic field-of-view adjustment based on the presented library data, an instruction to change at least one of the position, the attitude, or the field-of-view direction of the endoscope based on the library data; and candidate change processing of presenting, in a case where the user does not wish to execute the endoscopic field-of-view adjustment based on the presented library data, the user with information related to other library data classified in a same procedural scene as the library data being the execution candidate as another of the execution candidate, wherein the library data includes at least one relative parameter related to relative positions and attitudes of the endoscope and the observation object.

A second aspect of the present disclosure is a field-of-view control system comprising: the field-of-view control apparatus described above; the endoscope; a robot arm configured to change the position and the attitude of the endoscope; and a field-of-view direction changing unit configured to change the field-of-view direction of the endoscope, wherein the one or more processors being configured to control at least one of the robot arm or the field-of-view direction changing unit based on the presented library data.

A third aspect of the present disclosure is a field-of-view control method comprising determining a present procedural scene based on at least one of an endoscopic image of an observation object acquired by an endoscope, a position and an attitude of the endoscope, a field-of-view direction of the endoscope, or input information of the procedural scene by a user; presenting the user with information related to library data for realizing an endoscopic field of view associated with the determined procedural scene as an execution candidate for endoscopic field-of-view adjustment; changing, in a case where the user wishes to execute the endoscopic field-of-view adjustment based on the presented library data, at least one of the position, the attitude, or the field-of-view direction of the endoscope based on the library data; and presenting, in a case where the user does not wish to execute the endoscopic field-of-view adjustment based on the presented library data, the user with information related to other library data classified in a same procedural scene as the library data being the execution candidate as an other of the execution candidate, wherein the library data includes at least one relative parameter related to relative positions and attitudes of the endoscope and the observation object.

A fourth aspect of the present disclosure is a non-transitory computer-readable recording medium storing a field-of-view control program causing a computer to execute: scene determination processing of determining a present procedural scene based on at least one of an endoscopic image of an observation object acquired by an endoscope, a position and an attitude of the endoscope, a field-of-view direction of the endoscope, or input information of the procedural scene by a user; candidate presentation processing of presenting the user with information related to library data for realizing an endoscopic field of view associated with the determined procedural scene as an execution candidate for endoscopic field-of-view adjustment; field-of-view adjustment processing of changing, in a case where the user wishes to execute the endoscopic field-of-view adjustment based on the presented library data, at least one of the position, the attitude, or the field-of-view direction of the endoscope based on the library data; and candidate change processing of presenting, in a case where the user does not wish to execute the endoscopic field-of-view adjustment based on the presented library data, the user with information related to other library data classified in a same procedural scene as the library data being the execution candidate as another of the execution candidate, wherein the library data includes at least one relative parameter related to relative positions and attitudes of the endoscope and the observation object.

### BRIEF DESCRIPTION OF DRAWINGS

FIG. 1 is a schematic configuration diagram of a field-of-view control system according to a first embodiment of the present disclosure.

FIG. 2 is a diagram for describing a schematic configuration of an electric scope.

FIG. 3 is a diagram showing an example of a series of treatment steps.

FIG. 4 is a schematic configuration diagram of a field-of-view control apparatus.

FIG. 5 is a flow chart for describing a field-of-view control method according to the first embodiment of the present disclosure.

FIG. 6 is a diagram for describing a call of a library candidate by a processor according to the first embodiment of the present disclosure.

FIG. 7 is a diagram showing an example of a situation where a library candidate is presented to a user.

FIG. 8 is a diagram for describing a call of a library candidate by a processor according to a second embodiment of the present disclosure.

FIG. 9 is a flow chart for describing a field-of-view control method according to the second embodiment of the present disclosure.

FIG. 10 is a diagram for describing filtering by a processor.

FIG. 11 is a diagram for describing a call of a library candidate by a processor according to a third embodiment of the present disclosure.

FIG. 12 is a flow chart for describing a field-of-view control method according to the third embodiment of the present disclosure.

FIG. 13 is a diagram for describing ranking by a processor.

FIG. 14 is a diagram for describing a call of a library candidate by a processor in a mode when filtering and ranking are combined.

FIG. 15 is a diagram for describing filtering and ranking by a processor.

FIG. 16 is a diagram for describing a setting of a presentation order of library data.

FIG. 17A is a diagram for describing an example of calculations of filtering and ranking.

FIG. 17B is a diagram for describing an example of calculations of filtering and ranking.

FIG. 18 is a diagram for describing a call of a library candidate by a processor in a mode when prioritization and filtering are combined.

FIG. 19 is a diagram for describing ranking and filtering by a processor.

FIG. 20 is a diagram for describing a call of a library candidate by a processor according to a fourth embodiment of the present disclosure.

FIG. 21 is a diagram showing an example of a situation where information on a determined present procedural scene is presented to a user.

FIG. 22 is a diagram for describing a relationship between an inclination of an electric scope and an orientation of a camera.

FIG. 23 is a diagram for describing an orientation of an imaging optical system as viewed from a base coordinate.

FIG. 24 is a diagram for describing a relationship between an attitude of an observation object and an orientation of a camera.

FIG. 25 is a diagram for describing an orientation of an imaging optical system with respect to an observation object.

FIG. 26 is a diagram for describing a relationship between a direction in which an elongated observation object runs and an inclination around an optical axis of a camera.

FIG. 27 is a diagram showing an example of an endoscopic field of view in a state where a direction in which an elongated observation object runs faces a lateral direction with respect to a screen of a display.

FIG. 28 is a diagram for describing a method of adjusting a roll angle of an electric scope in accordance with a direction in which an elongated observation object runs.

FIG. 29 is a diagram for describing a method of adjusting a roll angle of an electric scope in accordance with an attitude of a patient or an operating table.

FIG. 30A is a diagram showing a relationship between a coordinate of an operating table or a patient and a base coordinate during normal time.

FIG. 30B is a diagram showing a relationship between a coordinate of an inclined operating table or a patient and an uncalibrated base coordinate.

FIG. 30C is a diagram showing a relationship between a coordinate of an inclined operating table or a patient and a calibrated base coordinate.

FIG. 31A is a diagram showing a relationship between a coordinate of an operating table or a patient and a base coordinate during normal time.

FIG. 31B is a diagram showing a relationship between a coordinate of an inclined operating table or a patient and an orientation of an electric scope when library data is not calibrated.

FIG. 31C is a diagram showing a relationship between a coordinate of an inclined operating table or a patient and an orientation of an electric scope when library data is calibrated.

FIG. 32 is a schematic configuration diagram of an oblique-viewing endoscope of a field-of-view control system according to a fifth embodiment of the present disclosure.

FIG. 33 is a diagram showing an oblique-viewing endoscope supported by a robot arm.

FIG. 34 is a diagram for describing that a field-of-view direction of an oblique-viewing endoscope changes around a longitudinal axis of a lens barrel unit.

FIG. 35 is a diagram for describing a look-up field of view of an oblique-viewing endoscope.

FIG. 36 is a diagram for describing a field of view when a curved mechanism of an electric scope is curved by around 30 degrees in an upward direction.

FIG. 37 is a diagram for describing a look-down field of view of an oblique-viewing endoscope.

FIG. 38 is a diagram for describing a field of view when a curved mechanism of an electric scope is curved by around 30 degrees in a downward direction.

FIG. 39 is a diagram for describing that a visual axis of a camera rotates around an axial line.

FIG. 40 is a schematic configuration diagram of a forward-viewing endoscope of a field-of-view control system according to a sixth embodiment of the present disclosure.

FIG. 41 is a diagram for describing that a visual axis of a camera rotates around an axial line.

FIG. 42 is a diagram showing a situation where an observation object is captured at a center of an endoscopic field of view.

FIG. 43 is a diagram showing a situation where an observation object is positioned at a center of a screen.

FIG. 44 is a diagram showing a situation where an observation object is captured at an end of an angle of view.

FIG. 45 is a diagram showing a situation where a location previously hidden from view becomes visible due to an endoscopic field of view of an angle of looking into an observation object.

### DESCRIPTION OF EMBODIMENTS

### First Embodiment

A field-of-view control apparatus, a field-of-view control system, a field-of-view control method, and a field-of-view control program according to a first embodiment of the present disclosure will be hereinafter described with reference to the drawings.

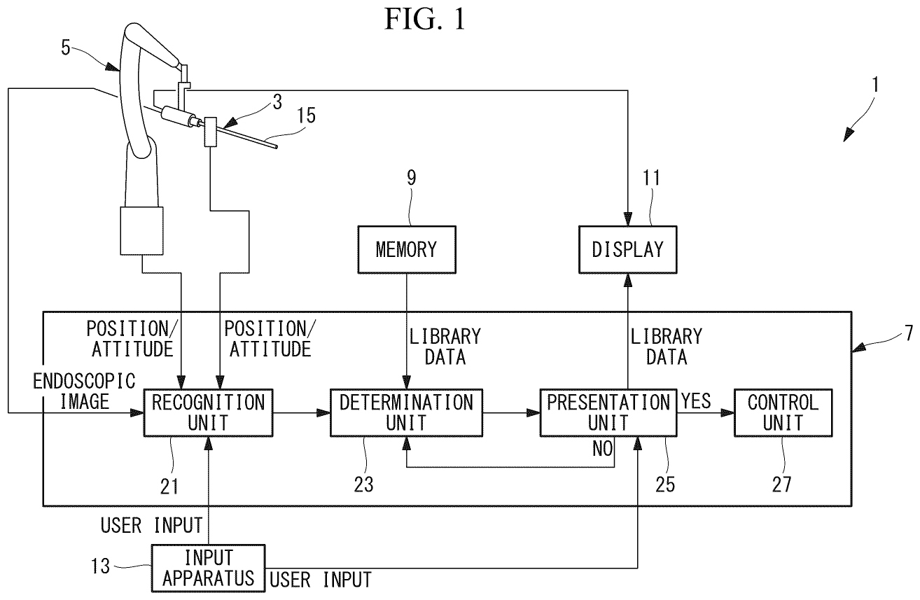

As shown in FIG. 1, a field-of-view control system 1 according to the present embodiment comprises an electric scope (endoscope) 3 that acquires an image of tissue inside a body cavity of a patient, a robot arm (position/attitude changing unit) 5 that supports the electric scope 3, a field-of-view control apparatus 7 that controls the electric scope 3 and the robot arm 5, a memory 9 that stores a database of library data for endoscope adjustment, a display 11 that displays an image and the like, and an input apparatus 13 that is a mouse, a keyboard, audio, a touch panel, and the like. By using the input apparatus 13, a user can input necessary instructions to the field-of-view control apparatus 7.

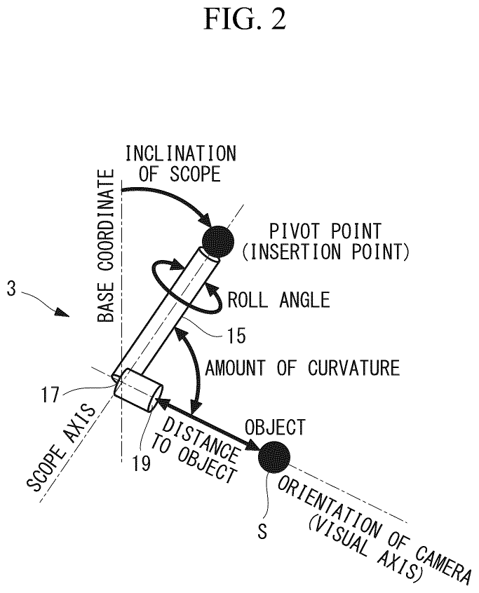

For example, as shown in FIG. 2, the electric scope 3 comprises an elongated insertion portion 15 that is equipped with a camera 19 at a tip portion thereof and a bending mechanism (field-of-view direction changing unit) 17 that causes an inclination angle of a field of view of the camera 19 to change. In addition, the electric scope 3 comprises a curved motor that drives the bending mechanism 17 and a roll motor (both motors not illustrated) that rolls and rotates the insertion portion 15 around a longitudinal axis. Furthermore, the electric scope 3 has a ranging function of measuring a distance to an observation object S such as a blood vessel captured in a field of view of the camera 19. Known mechanisms can be adopted as a mechanism of realizing the ranging function.

For example, the camera 19 comprises at least one lens and an imaging element such as a CCD (Charge Coupled Device) or a CMOS (Complementary Metal-Oxide-Semiconductor). The camera 19 may be a monocular camera or a stereo camera. The camera 19 is provided with a rotation mechanism (position/attitude changing unit) for adjusting an angle of the imaging element and a motor (not illustrated) that drives the rotation mechanism.

The robot arm 5 is, for example, an electric holder of a general-purpose 6-axis articulated robot that movably holds the electric scope 3 at any position. The robot arm 5 comprises, for each joint, a motor (not illustrated) that operates each joint.

The memory 9 stores, in association with each procedural scene, a plurality of pieces of library data for realizing an endoscopic field of view of the electric scope 3 that are associated with each procedural scene. The library data includes at least one relative parameter (library parameter) related to relative positions and attitudes of the electric scope 3 and the observation object S.

Examples of the relative parameter include scope-shaft roll angle information, distance information on a distance between the camera 19 of the electric scope 3 and the observation object S, and amount-of-curvature information on an amount of curvature of the bending mechanism 17 of the electric scope 3. The scope-shaft roll angle information represents a roll angle from an initial state around a longitudinal axis of the insertion portion 15 of the electric scope 3. The distance information on the distance between the camera 19 of the electric scope 3 represents a distance between the camera 19 and the observation object S in a direction along a visual axis of the camera 19. The amount-of-curvature information on the amount of curvature of the bending mechanism 17 of the electric scope 3 represents an orientation of the camera 19 with respect to a scope shaft of the electric scope 3. Hereinafter, these pieces of information will be referred to as roll angle information, distance information, and amount-of-curvature information.

In other words, in the memory 9, an endoscopic field of view suitable for each procedural scene has been set in advance for each procedural scene and library data including at least any one of roll angle information, distance information, and amount-of-curvature information for realizing each endoscopic field of view is stored in association with each procedural scene.

The field-of-view control apparatus 7 comprises a recognition unit 21 that determines a present procedural scene, a determination unit 23 that selects library data as an execution candidate for endoscopic field-of-view adjustment, a presentation unit 25 that presents the user with information related to the selected library data, and a control unit 27 that controls a position and an attitude of the electric scope 3 based on the library data.

The recognition unit 21 determines the present procedural scene based on at least one of the endoscopic image acquired by the electric scope 3, the position and the attitude of the electric scope 3, or the input information of the procedural scene by the user. For example, a procedural scene is classified according to a specific operation by an operator, an anatomical feature on a screen, and a treatment tool on the screen.

Examples of the specific operation by an operator include dissection, incision, expansion, and hemostasis. Examples of the anatomical feature on a screen include Merkmal and anatomical structures. Examples of Merkmal include main blood vessels and branches thereof, ureters, biliary ducts, and marked tumors. Examples of anatomical structures include lymph nodes, mesenteric depressions, running of intestines, nerve plexus, and connective tissue layers. Examples of the treatment tool on the screen include a type of the treatment tool, a motion such as opening or closing, an amount by which the treatment tool is grasped, and a state of an object being handled. Examples of the type of the treatment tool include monopolar or bipolar, a grasper or a dissector, and a needle-holder or not.

For example, the recognition unit 21 determines the present procedural scene by recognizing a feature such as a treatment tool, a blood vessel, and an organ from an endoscopic image. In addition, the recognition unit 21 determines the present procedural scene by estimating the position, the attitude, and the field-of-view direction of the electric scope 3 from a numerical value of each of the motors of the electric scope 3 and the robot arm 5. Furthermore, the recognition unit 21 determines a procedural scene inputted by the user as the present procedural scene.

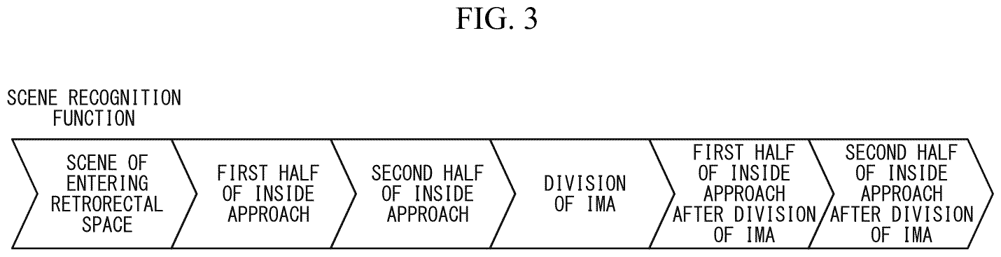

In the present embodiment, a flow of procedures may be programmed in advance and switching among pieces of library data associated with each procedural scene may be performed in accordance with a treatment step or, in other words, a procedural scene. In this case, for example, the recognition unit 21 may switch the present procedural scene to another procedural scene by having an AI (Artificial Intelligence) determine a series of treatment steps such as those shown in FIG. 3.

A procedural scene is, for example, an anatomical location being treated or an operation by an operator such as an incision operation. An AI may estimate a treatment step based on the anatomical location or an operation by the operator such as an incision. In addition to an incision operation, examples of an operation by an operator include an expansion operation in which the operator expands tissue including an affected area in any direction using forceps, a dissection operation in which tissue including an affected area is dissected from tissue to be retained by cutting the tissue with an energy treatment tool such as an electrocautery scalpel, a hemostatic operation with respect to a division or hemorrhage of a specific blood vessel, clipping of a vascular channel, a pressure removal operation of an organ, and a surgical operation such as fluorescent observation.

In addition, a treatment step may be recognized by the operator. For example, a treatment step may be specified by the operator by using a UI (User Interface) such as audio or a button operation. In this case, for example, the recognition unit 21 may switch among pieces of library data based on the operator specifying a treatment step by UI. Furthermore, for example, the recognition unit 21 may determine a designated procedural scene based on an instruction by the operator such as “Proceed to next treatment step”, “Return to previous treatment step”, or “Proceed to step of division of the IMA (Inferior Mesenteric Artery)”.

The determination unit 23 extracts library data associated with the present procedural scene determined by the recognition unit 21 by referring to the database stored in the memory 9. When there are a plurality of pieces of library data classified into a same procedural scene, the determination unit 23 randomly extracts any one piece of library data excluding library data that has already been presented.

In addition, when the user makes a selection to the effect that the user does not wish to execute endoscopic field-of-view adjustment based on the extracted library data, the determination unit 23 extracts another piece of library data classified into the same procedural scene as the extracted piece of library data from the database in the memory 9.

The presentation unit 25 displays information related to the library data extracted by the determination unit 23 as an execution candidate for endoscopic field-of-view adjustment on the display 11. Examples of the information related to the library data include numerical information representing at least one of the position, the attitude, or the field-of-view direction of the electric scope 3 when endoscopic field-of-view adjustment is executed based on the library data, a schematic view of an endoscopic image, a schematic view showing positions and attitudes of the electric scope 3 and the robot arm 5, and an endoscopic image acquired in a past case. Examples of the numerical information representing the position and the attitude of the electric scope 3 include a group of parameters such as a roll angle, a pitch angle, and a yaw angle of the electric scope 3, an amount of insertion of the insertion portion 15, and an angle of curvature of the bending mechanism 17 of the electric scope 3. In addition, the presentation unit 25 accepts an input from the user with respect to whether or not the user wishes to execute endoscopic field-of-view adjustment based on the library data displayed on the display 11.

For example, the schematic view of an endoscopic image may be an image obtained by converting an endoscopic image or the like acquired in a past case into a schematic image by image processing or an image generated as an endoscopic image that is predicted after executing an execution-candidate library by an AI (Artificial Intelligence) of an image generating system using relative parameters of the execution-candidate library and the present position and attitude and/or the field-of-view direction of the electric scope 3.

When displaying numerical information as information related to library data, since the amount of information to be processed is smaller compared to when displaying an image, display speed can be increased.

In addition, when displaying a schematic view of an endoscopic image as information related to library data, features of the field of view can be displayed in a more comprehensible manner as compared to an actual endoscopic image. Therefore, the user can intuitively comprehend an image of the field of view after executing field-of-view adjustment.

In addition, displaying a schematic view indicating positions and attitudes of the electric scope 3 and the robot arm 5 as information related to library data enables the user to imagine a positional relationship between the pair made up of the electric scope 3 and the robot arm 5 and the user during field-of-view adjustment and promotes smooth progress of procedures.

Furthermore, adopting an endoscopic image acquired in a past case as information related to library data has an advantage of making a minute difference in a field of view between pieces of library data more comprehensible to the user.

When the user makes a selection to the effect that the user wishes to execute endoscopic field-of-view adjustment based on the library data presented by the presentation unit 25, the control unit 27 outputs an instruction to change the position and the attitude of the electric scope 3 based on the library data to the electric scope 3 and the robot arm 5. Accordingly, the position and the attitude of the electric scope 3 are adjusted due to at least any one of the bending mechanism 17 of the electric scope 3, the rotation mechanism of the camera 19, and each joint of the robot arm 5 being controlled.

For example, when the presented library data includes distance information as a relative parameter, the control unit 27 causes an actual distance between the camera 19 of the electric scope 3 and the observation object S that is calculated from image information of the observation object S to match the distance information by controlling at least one of the bending mechanism 17 of the electric scope 3 or the robot arm 5.

Specifically, the control unit 27 calculates a trajectory for matching the distance between the camera 19 of the electric scope 3 and the observation object S with the distance information by comparing a present measured distance value to the observation object S and the distance information included in the library data. The present measured distance value to the observation object S is measured by the ranging function of the electric scope 3.

Next, based on the calculated trajectory, the control unit 27 uses inverse kinematics of the robot arm 5 to determine a drive amount of each joint of the robot arm 5 required to match the distance between the camera 19 and the observation object S with the distance information. The determined drive amount of each joint is inputted to each motor of the robot arm 5 as an angle command for each joint. In addition, based on the calculated trajectory, the control unit 27 determines an angle of curvature of the bending mechanism 17 required to match the distance between the camera 19 and the observation object S with the distance information. The determined angle of curvature of the bending mechanism 17 is inputted to the motor of the electric scope 3 as a motor angle command.

Due to each joint of the robot arm 5 moving according to each angle command and the bending mechanism 17 of the electric scope 3 curving according to the motor angle command, an endoscopic field of view in which the distance between the camera 19 of the electric scope 3 and the observation object S matches the distance information is obtained.

In addition, for example, when the presented library data includes amount-of-curvature information as a relative parameter, the control unit 27 causes an orientation of the camera 19 of the electric scope 3 with respect to the observation object S that is calculated from image information of the observation object S to match the amount-of-curvature information by controlling at least one of the bending mechanism 17 of the electric scope 3 or the robot arm 5.

Specifically, the control unit 27 calculates a trajectory for matching the amount of curvature of the bending mechanism 17 with the amount-of-curvature information by comparing a present amount of curvature of the bending mechanism 17 with the amount-of-curvature information included in the library data.

Next, based on the calculated trajectory, the control unit 27 uses inverse kinematics of the robot arm 5 to determine a drive amount of each joint of the robot arm 5 required to match the amount of curvature of the bending mechanism 17 with the amount-of-curvature information. The determined drive amount of each joint is inputted to each motor of the robot arm 5 as an angle command for each joint. In addition, based on the calculated trajectory, the control unit 27 determines an angle of curvature of the bending mechanism 17 required to match the amount of curvature of the bending mechanism 17 with the amount-of-curvature information. The determined angle of curvature of the bending mechanism 17 is inputted to the motor of the electric scope 3 as a motor angle command.

Due to each joint of the robot arm 5 moving according to each angle command and the bending mechanism 17 of the electric scope 3 curving according to the motor angle command, an endoscopic field of view in which the orientation of the camera 19 of the electric scope 3 with respect to the observation object S matches the amount-of-curvature information is obtained.

In addition, for example, when the presented library data includes roll angle information, the control unit 27 causes an inclination of the observation object S around an optical axis of the camera 19 of the electric scope 3 that is calculated from image information of the observation object S to match the roll angle information by controlling the robot arm 5.

Specifically, by comparing a present roll angle of the scope shaft and the roll angle information, the control unit 27 determines variations of a position and an attitude around a pivot point required to match the roll angle of the scope shaft with the roll angle information. For example, the determined variations of the position and the attitude around the pivot point includes the roll angle, the pitch angle, the yaw angle, and an amount of forward/backward movement of the electric scope 3.

Based on the determined variations of the position and the attitude around the pivot point, the control unit 27 uses inverse kinematics of the robot arm 5 to determine a drive amount of each joint required to match the roll angle of the scope shaft with the roll angle information. The determined drive amount of each joint is inputted to each motor of the robot arm 5 as an angle command for each joint. Due to each joint of the robot arm 5 moving according to each angle command, an endoscopic field of view in which an inclination of the observation object S around the optical axis of the camera 19 of the electric scope 3 matches the roll angle information is obtained.

When controlling the robot arm 5, the control unit 27 calculates Euler angles (roll, pitch, and yaw) based on the angle of each joint using forward kinematics of the robot arm 5. Accordingly, the present roll angle of the scope shaft is calculated. The calculated present roll angle of the scope shaft is stored by the control unit 27.

When controlling the curved mechanism 17 of the electric scope 3, the control unit 27 calculates a present amount of curvature of the bending mechanism 17 by converting a motor angle of the bending mechanism 17 into an amount of curvature. The calculated present amount of curvature is stored by the control unit 27.

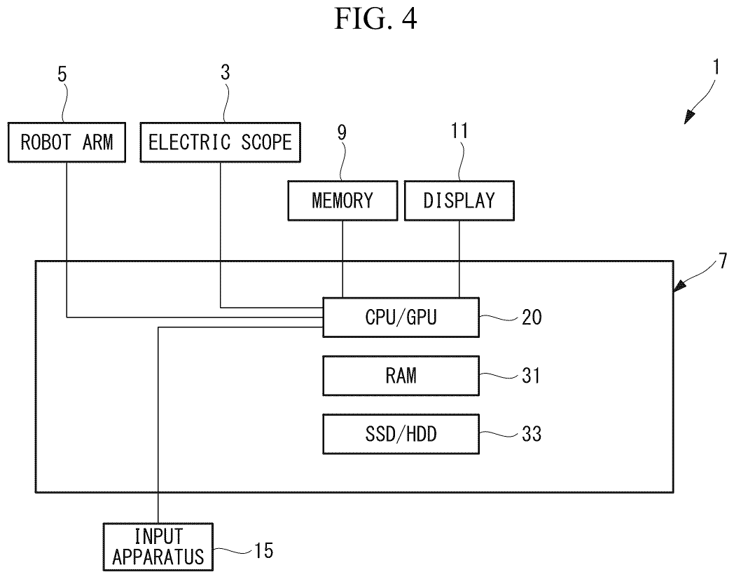

The field-of-view control apparatus 7 described above is realized by, for example, a dedicated computer or a general-purpose computer. In other words, as shown in FIG. 4, the field-of-view control apparatus 7 comprises a processor 20 that is a CPU (Central Processing Unit) or a GPU (Graphics Processing Unit) constituting the recognition unit 21, the determination unit 23, the presentation unit 25, and the control unit 27, a main storage apparatus 31, and an auxiliary storage apparatus 33.

The main storage apparatus 31 is a RAM (Random Access Memory) or the like to be used as a work area of the processor 20.

The auxiliary storage apparatus 33 is a non-transitory computer-readable recording medium such as an SSD (Solid State Drive) or an HDD (Hard Disk Drive). The auxiliary storage apparatus 33 stores a field-of-view control program that causes the processor 20 to execute processing. The main storage apparatus 31 and the auxiliary storage apparatus 33 may be configured to be connected to the field-of-view control apparatus 7 via a network.

The field-of-view control program causes the processor 20 to execute: scene determination processing of determining a present procedural scene; candidate presentation processing of presenting the user with information related to library data associated with the determined procedural scene as an execution candidate for endoscopic field-of-view adjustment; field-of-view adjustment processing of changing, when the user wishes to execute endoscopic field-of-view adjustment based on the presented library data, at least one of the position, the attitude, or the field-of-view direction of the electric scope 3 based on the library data; and candidate change processing of presenting, when the user does not wish to execute endoscopic field-of-view adjustment based on the presented library data, the user with information related to other library data classified in the same procedural scene as the library data being the execution candidate as another of the execution candidate to the user.

Each of the functions described above of the recognition unit 21, the determination unit 23, the presentation unit 25, and the control unit 27 is realized due to the processor 20 executing processing according to the field-of-view control program. Hereinafter, each processing step described above performed by the processor 20 will be described as a processing step performed by the recognition unit 21, the determination unit 23, the presentation unit 25, or the control unit 27.

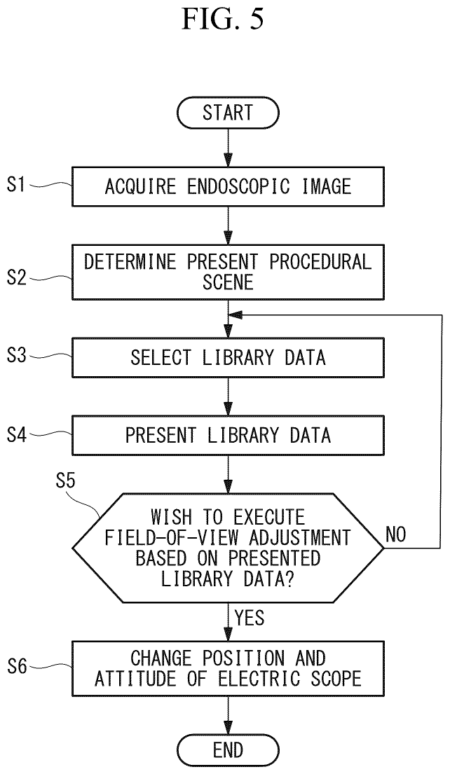

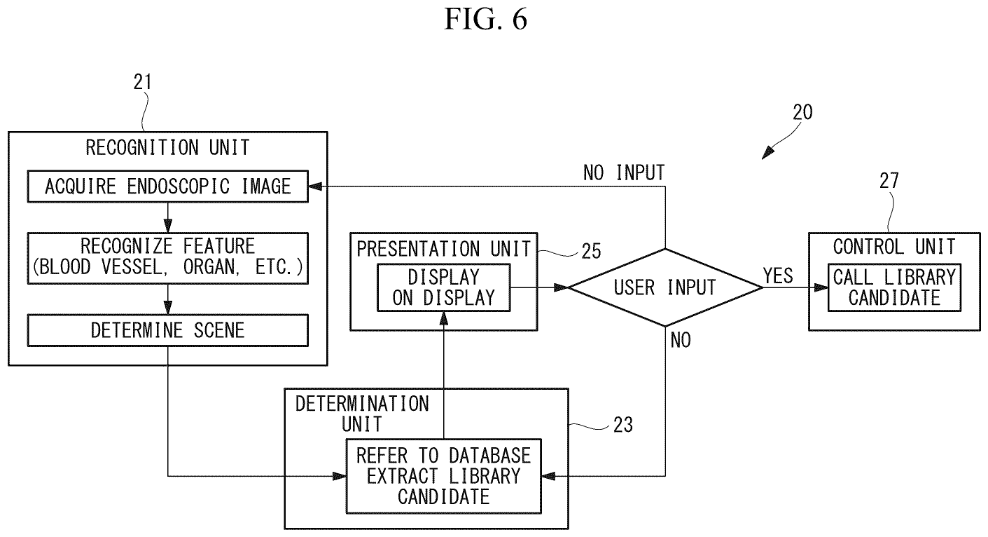

Next, effects of the field-of-view control apparatus 7, the field-of-view control system 1, the field-of-view control method, and the field-of-view control program configured as described above will be described with reference to the flow chart in FIG. 5 and FIG. 6.

When performing an endoscopic procedure using the field-of-view control apparatus 7, the field-of-view control system 1, the field-of-view control method, and the field-of-view control program according to the present embodiment, first, the insertion portion 15 of the electric scope 3 is inserted into the body of a patient. In addition, due to the camera 19 of the insertion portion 15 photographing the observation object S that is a blood vessel or the like, an endoscopic image of the observation object S is acquired (step S1). The acquired endoscopic image is displayed on the display 11 and inputted to the recognition unit 21 of the field-of-view control apparatus 7.

Next, the recognition unit 21 determines the present procedural scene based on at least one of the endoscopic image inputted from the electric scope 3, the position and the attitude of the electric scope 3, or the input information of the procedural scene by the user (step S2). In the example shown in FIG. 6, the present procedural scene is determined by the recognition unit 21 due to features such as a blood vessel, an organ, and the like being recognized in the endoscopic image from the electric scope 3.

Next, the determination unit 23 randomly extracts one piece of library data associated with the present procedural scene determined by the recognition unit 21 by referring to the database stored in the memory 9 (step S3). The library data extracted by the determination unit 23 is also referred to as a library candidate.

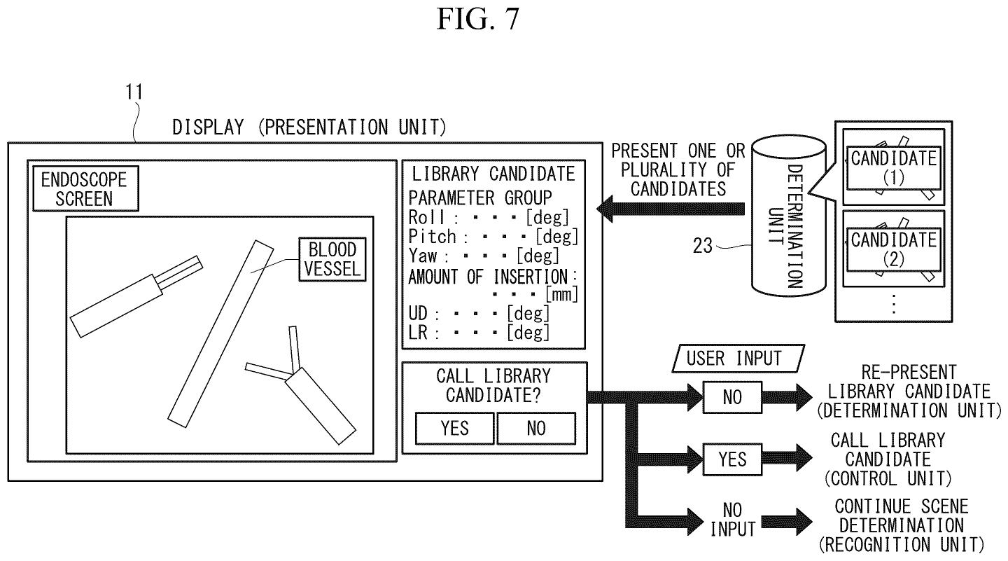

Next, for example, as shown in FIG. 7, the presentation unit 25 displays information related to the library candidate extracted by the determination unit 23 as an execution candidate for endoscopic field-of-view adjustment on the display 11 (step S4). In the example shown in FIG. 7, a parameter group including the roll angle (deg), the pitch angle (deg), and the yaw angle (deg) of the electric scope 3, an amount of insertion (mm) of the insertion portion 15, and UD (up/down, deg) and LR (left/right, deg) of the bending mechanism 17 of the electric scope 3 is shown as the library candidate.

Next, the user makes a selection with respect to whether or not the user wishes to execute endoscopic field-of-view adjustment based on the library candidate displayed on the display 11 (step S5). In the example shown in FIG. 7, when the user wishes to execute, the user inputs (YES) for calling the presented library candidate, but when the user does not wish to execute, the user inputs (NO) for not calling the presented library candidate. When wishing to execute or, in other words, when an input to the effect that the library candidate is to be called is made, the control unit 27 calls the presented library candidate.

In addition, the control unit 27 outputs an instruction to change the position and the attitude of the electric scope 3 based on the called library candidate to the robot arm 5 and the electric scope 3 (step S6). Accordingly, due to the position and the attitude of the electric scope 3 being adjusted, an endoscopic field of view desired by the user is displayed on the display 11.

On the other hand, when not wishing to execute or, in other words, when an input to the effect that the library candidate is not to be called is made, a return is made to step 3. In addition, the determination unit 23 randomly extracts one of the other library candidates classified into the same procedural scene as the library candidate being displayed on the display 11 (step S3).

Next, the presentation unit 25 displays information related to the library candidate newly extracted by the determination unit 23 as another execution candidate for endoscopic field-of-view adjustment on the display 11 (step S4). In addition, the presentation unit 25 accepts an input from the user with respect to the new library candidate displayed on the display 11.

Step S3 to step S5 are repeated until the user makes a selection to the effect that the user wishes to execute endoscopic field-of-view adjustment based on the library candidate displayed on the display 11. Accordingly, the user can select a more preferable library candidate and an endoscopic field of view desired by the user can be provided.

As shown in FIG. 6 and FIG. 7, when there is no input from the user even after a predetermined amount of time elapses in step S5, a return is made to step S2. In addition, a determination of a procedural scene by the recognition unit 21 (step S2), an extraction of a library candidate by the determination unit 23 (step S3), and a presentation of the library candidate by the presentation unit 25 (step S4) are performed. Accordingly, the library candidate displayed on the display 11 is updated in real time. The user makes a selection on whether or not the user wishes to execute endoscopic field-of-view adjustment with respect to the updated library candidate (step S5).

As described above, according to the field-of-view control apparatus 7, the field-of-view control system 1, the field-of-view control method, and the field-of-view control program according to the present embodiment, library data suitable for the present procedural scene is presented to the user as an execution candidate of endoscopic field-of-view adjustment by the processor 20 of the field-of-view control apparatus 7. In addition, the presented library data is changed to a next candidate according to a selection made by the user. Furthermore, endoscopic field-of-view adjustment is executed based on the library data selected by the user.

Therefore, the user can call a desired endoscopic field of view in accordance with a procedural scene by a simple operation of merely selecting, according to the user's preference or according to a case, library data presented by the processor without having to perform manual adjustment. In addition, since the user need only issue, using the input apparatus 15, an instruction on whether or not the user wishes to execute endoscopic field-of-view adjustment based on the library candidate presented by the processor 20, an amount of verbal instructions by the user can be reduced.

Furthermore, while conventional endoscopic procedures have a possibility that increasing a degree of freedom of endoscopic operations in order to provide an operator with a better endoscopic field of view may end up increasing the number of determinations to be made by a scopist and the number of instructions to be issued from the operator to the scopist due to a wider variety of adjustment parameters, adopting the field-of-view control apparatus 7, the field-of-view control system 1, the field-of-view control method, and the field-of-view control program according to the present embodiment enables determinations by the operator and the scopist to be assisted and leads to reductions in surgical operating time and labor.

### Second Embodiment

Next, the field-of-view control apparatus 7, the field-of-view control system 1, a field-of-view control method, and a field-of-view control program according to a second embodiment of the present disclosure will be described.

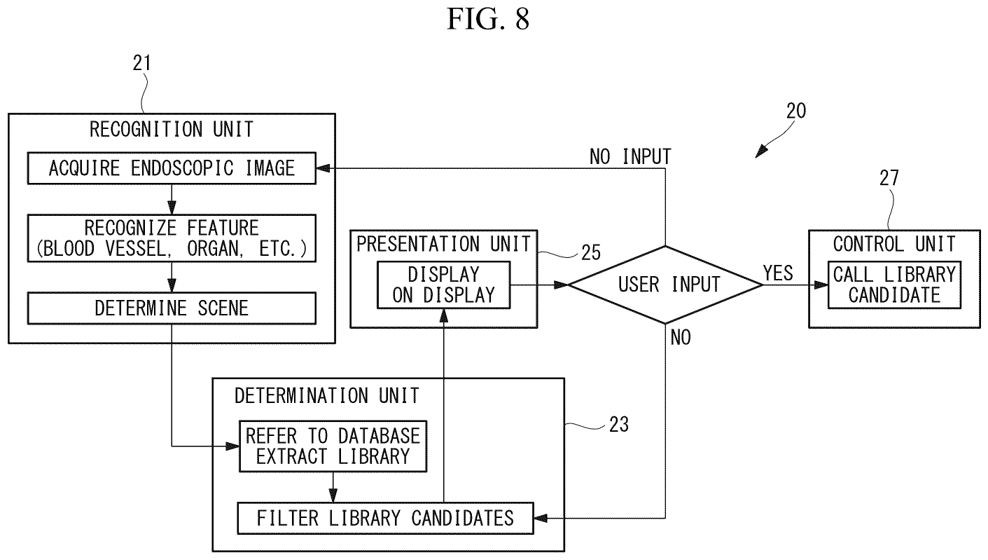

The field-of-view control apparatus 7, the field-of-view control system 1, the field-of-view control method, and the field-of-view control program according to the present embodiment differ from the first embodiment in that, for example, as shown in FIG. 8, the processor 20 executes filtering for narrowing down library data to be presented as an execution candidate for endoscopic field-of-view adjustment.

Hereinafter, portions that share common configurations with the field-of-view control apparatus 7, the field-of-view control system 1, the field-of-view control method, and the field-of-view control program according to the first embodiment will be assigned the same reference signs and descriptions thereof will be omitted.

The memory 9 stores a plurality of pieces of library data in a state where each piece of library data is associated with tag information representing an attribute or a feature in addition to a procedural scene. As examples of tag information, information related to the user, information related to a facility to be used by the field-of-view control system 1, information related to a patient, information related to a type of treatment tool, information related to a type of the electric scope 3, information related to a case such as whether the case is a difficult case or not, information on library data, performance of surgery such as a volume of hemorrhage and a surgical operating time, and the like are assumed. Examples of the information related to the user include a name, an ID, gender, age, and an affiliated facility. Examples of the information related to a facility include a facility name, an area, and what procedural processes are being adopted. Examples of the information related to a patient include a name, an ID, gender, age, and progress. Examples of information on library data include date and time of registration of the library data, date and time of execution of the library data, and frequency of execution of the library data.

The user sets a refinement condition based on tag information in advance or during a procedure using the input apparatus 13. Examples of the refinement condition include conditions such as narrowing down to only library data associated with specific tag information, narrowing down to only library data associated with tag information within a specific quantitative numerical range, narrowing down to only library data associated with tag information within a numerical range calculated by a specific calculation formula from a plurality of pieces of tag information, and not narrowing down based on tag information.

Examples of library data associated with specific tag information include library data linked to the user as an individual such as library data previously registered by the user, library data linked to the affiliated facility of the user such as library data registered at a facility the user is affiliated with, and library data linked to all system users such as all of the pieces of library data registered by the users of the field-of-view control system 1.

Examples of library data associated with tag information within a specific quantitative numerical range include library data of a past case of which a volume of hemorrhage or a surgical operating time is within a predetermined range and library data of which a date and time of registration is within a predetermined period.

After extracting a plurality of library candidates associated with the present procedural scene determined by the recognition unit 21 from the database in the memory 9, the determination unit 23 narrows down library candidates to be extracted as an execution candidate according to the refinement condition based on the tag information designated by the user. When there are a plurality of narrowed-down library candidates, one of the library candidates may be randomly extracted.

In addition, when the user makes a selection to the effect that the user does not wish to execute endoscopic field-of-view adjustment based on the extracted library candidate, the determination unit 23 may randomly extract any other library candidate from the plurality of narrowed-down library candidates. When all of the narrowed-down library candidates have already been presented, the user may be notified of a message to that effect via the presentation unit 25.

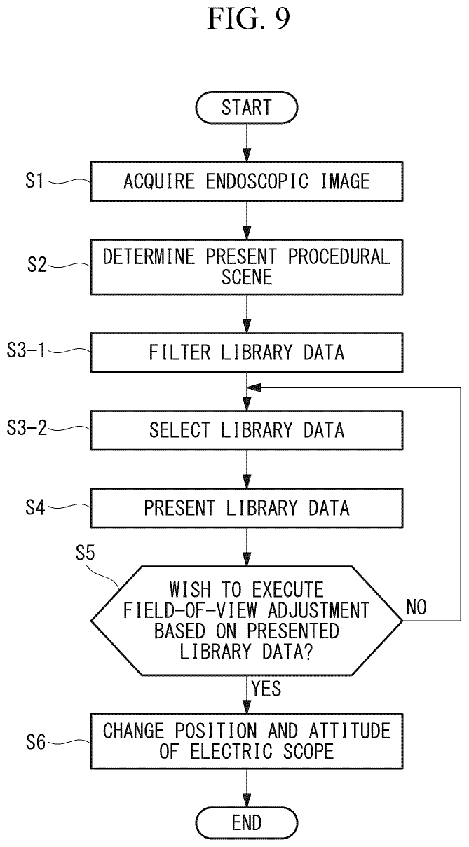

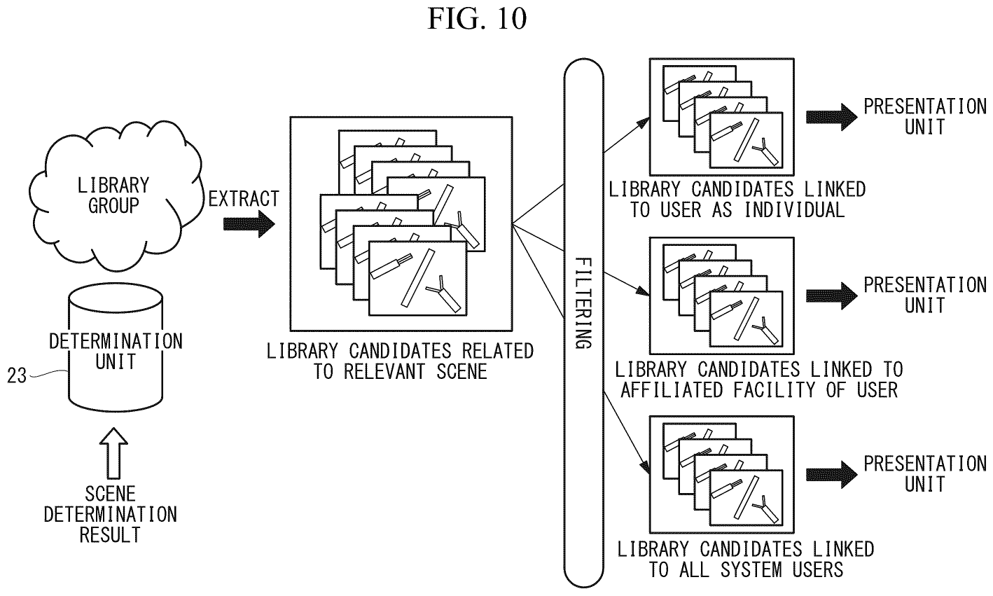

Next, effects of the field-of-view control apparatus 7, the field-of-view control system 1, the field-of-view control method, and the field-of-view control program configured as described above will be described with reference to the flow chart in FIG. 9 and FIG. 10.

When performing an endoscopic procedure, the user sets a refinement condition based on tag information in advance or during the procedure.

When the recognition unit 21 determines the present procedural scene (step S2), the determination unit 23 extracts a plurality of library candidates associated with the present procedural scene from the database stored in the memory 9.

Next, the determination unit 23 narrows down the extracted plurality of library candidates according to the set refinement condition (step S3-1). In addition, the determination unit 23 extracts one library candidate to be presented as an execution candidate from the plurality of narrowed-down library candidates (step S3-2). The extracted library candidate is displayed on the display 11 by the presentation unit 25 (step S4).

FIG. 10 shows an example of narrowing down to library candidates linked to the user as an individual by filtering, an example of narrowing down to library candidates linked to an affiliated facility of the user by filtering, and an example of narrowing down to library candidates linked to all system users by filtering. One refinement condition may be set according to the user's preference, a case, or the like.

When the user performs an input to the effect that the user does not wish to execute endoscopic field-of-view adjustment based on the library candidate displayed on the display 11 in step S5, a return is made to step S3-2. In addition, after the determination unit 23 randomly extracts any other library candidate from the plurality of narrowed-down library candidates (step S3-2), the presentation unit 25 displays the extracted library candidate on the display 11 (step S4).

As described above, according to the field-of-view control apparatus 7, the field-of-view control system 1, the field-of-view control method, and the field-of-view control program according to the present embodiment, based on an attribute or a feature of library data, the processor 20 narrows down library data required by the user among a plurality of pieces of library data classified into a same procedural scene. Therefore, library data desired by the user can be called more quickly and accurately. This is particularly effective when there are a large number of pieces of library data classified into a same procedural scene.

### Third Embodiment

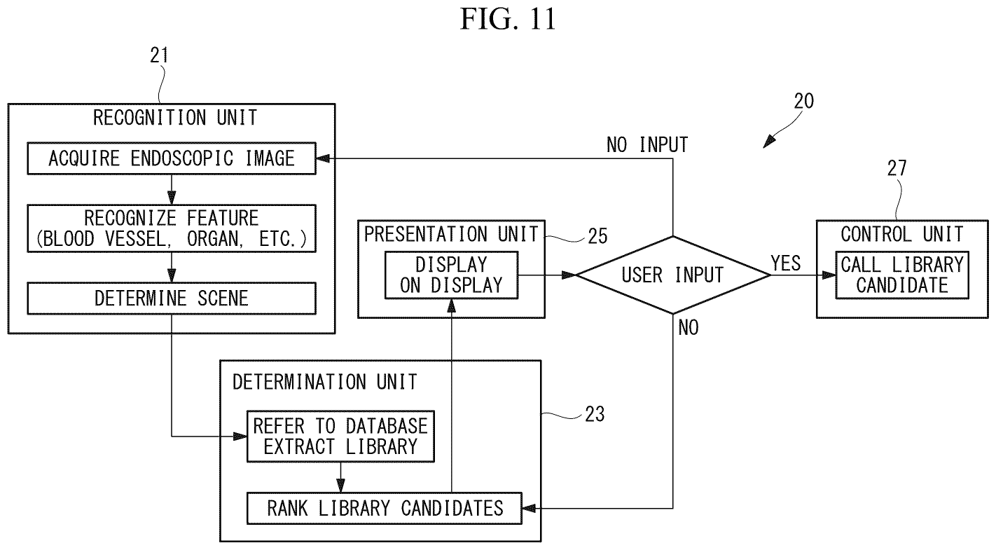

Next, the field-of-view control apparatus 7, the field-of-view control system 1, a field-of-view control method, and a field-of-view control program according to a third embodiment of the present disclosure will be described. The field-of-view control apparatus 7, the field-of-view control system 1, the field-of-view control method, and the field-of-view control program according to the present embodiment differ from the second embodiment in that, for example, as shown in FIG. 11, the processor 20 executes ranking of assigning an order of priority to library data to be presented as an execution candidate for endoscopic field-of-view adjustment instead of filtering.

Hereinafter, portions that share common configurations with the field-of-view control apparatus 7, the field-of-view control system 1, the field-of-view control method, and the field-of-view control program according to the second embodiment will be assigned the same reference signs and descriptions thereof will be omitted.

The memory 9 stores library data in a state where the library data is associated with tag information representing an attribute or a feature in addition to a procedural scene. The tag information is similar to that according to the second embodiment.

The user sets an order of priority based on tag information in advance or during a procedure using the input apparatus 13. Examples of setting an order of priority include prioritization based on a magnitude of a numerical value calculated by a specific calculation formula from a plurality of pieces of tag information and prioritization based on specific quantitative numerical values of tag information. Examples of prioritization based on specific quantitative numerical values of tag information include prioritizing library data in a descending order of recentness of registration or use in a time series and prioritizing library data in a descending order of frequency of adoption of call.

After extracting a plurality of library candidates associated with the present procedural scene determined by the recognition unit 21 from the database in the memory 9, the determination unit 23 prioritizes library candidates to be extracted as an execution candidate according to the order of priority based on tag information designated by the user.

In addition, when the user makes a selection to the effect that the user does not wish to execute endoscopic field-of-view adjustment based on the extracted library candidate, the determination unit 23 extracts a library candidate with a next highest order of priority to the library candidate that has already been extracted. When all of the prioritized library candidates have already been presented, the user may be notified of a message to that effect via the presentation unit 25.

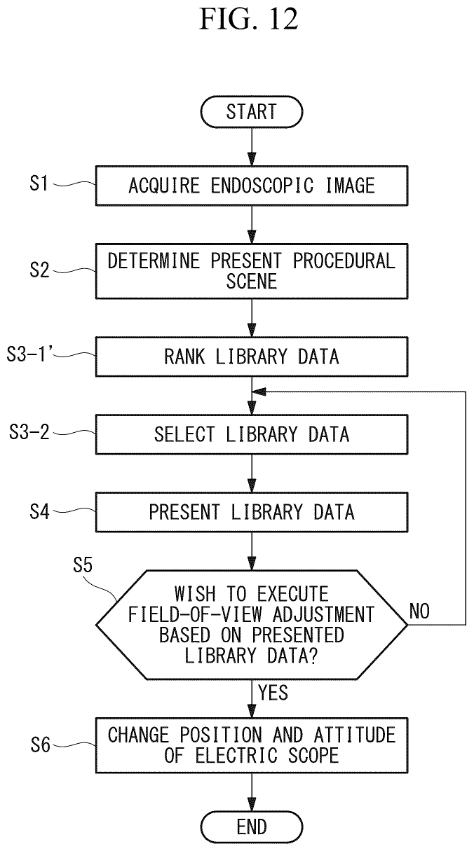

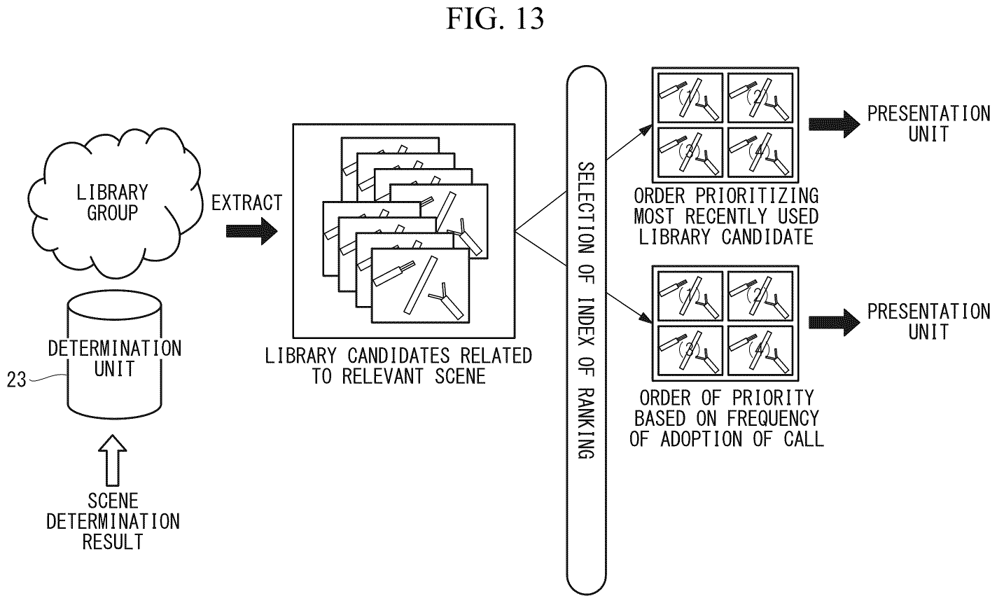

Next, effects of the field-of-view control apparatus 7, the field-of-view control system 1, the field-of-view control method, and the field-of-view control program configured as described above will be described with reference to the flow chart in FIG. 12 and FIG. 13.

When performing an endoscopic procedure, the user sets an order of priority based on tag information in advance or during the procedure.

When the recognition unit 21 determines the present procedural scene (step S2), the determination unit 23 extracts a plurality of library candidates associated with the present procedural scene from the database stored in the memory 9.

Next, the determination unit 23 prioritizes the extracted plurality of library candidates according to the set order of priority (step S3-1′). In addition, the determination unit 23 extracts the library candidate with the highest order of priority as an execution candidate (step S3-2). The extracted library candidate is displayed on the display 11 by the presentation unit 25 (step S4).

FIG. 13 shows an example of preferentially extracting a library candidate used most recently by ranking and an example of preferentially extracting a library candidate with a high frequency of adoption of call by ranking. One order of priority may be set according to the user's preference, a case, or the like.

When the user performs an input to the effect that the user does not wish to execute endoscopic field-of-view adjustment based on the library candidate displayed on the display 11 in step S5, a return is made to step S3-2. In addition, after the determination unit 23 extracts the library candidate with the next highest order of priority to the library candidate that has already been extracted (step S3-2), the presentation unit 25 displays the extracted library candidate on the display 11 (step S4).

As described above, according to the field-of-view control apparatus 7, the field-of-view control system 1, the field-of-view control method, and the field-of-view control program according to the present embodiment, based on an attribute or a feature of library data, the processor 20 ranks, in compliance with the user's request, a plurality of pieces of library data classified into a same procedural scene. Therefore, library data desired by the user can be called more quickly and efficiently. This is also particularly effective when there are a large number of pieces of library data classified into a same procedural scene.

The filtering according to the second embodiment and the ranking according to the third embodiment can be appropriately combined.

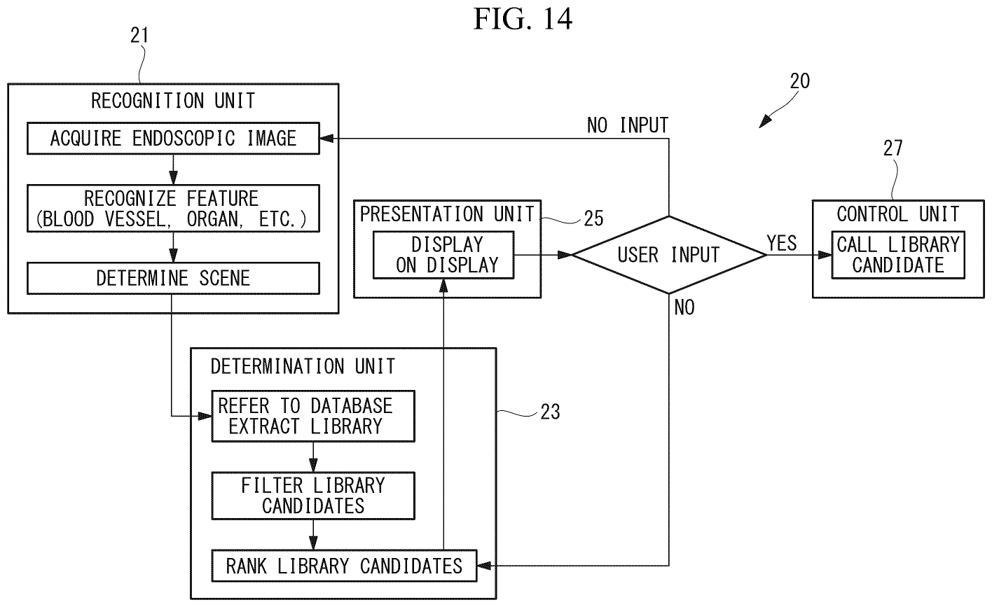

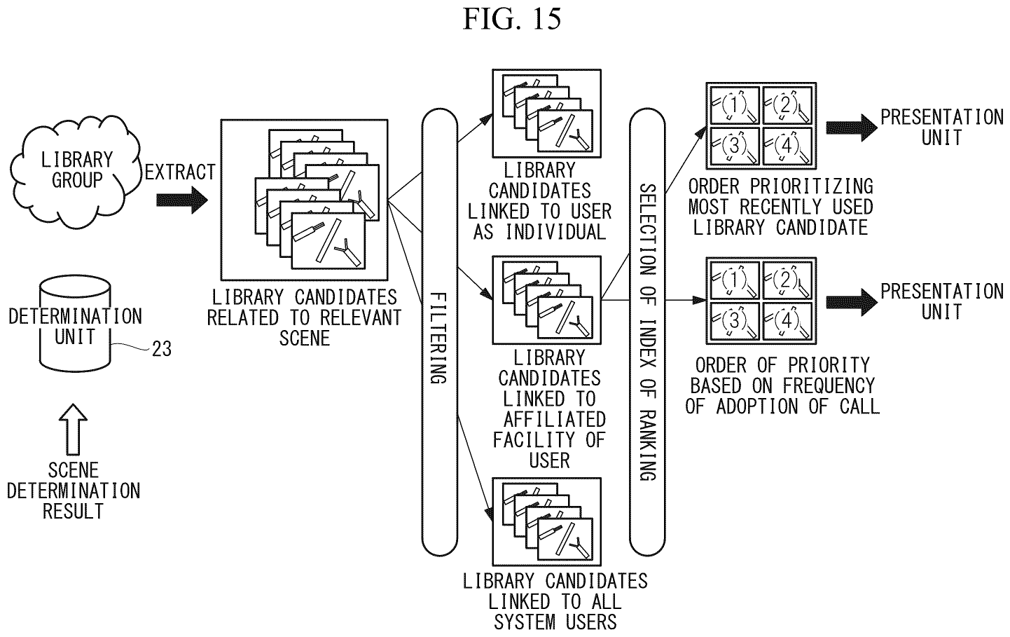

For example, as shown in FIG. 14 and FIG. 15, after narrowing down library candidates to be presented as an execution candidate according to a refinement condition designated by the user from a plurality of extracted library candidates, the determination unit 23 may prioritize the narrowed-down library candidates to be selected as an execution candidate according to an order of priority designated by the user.

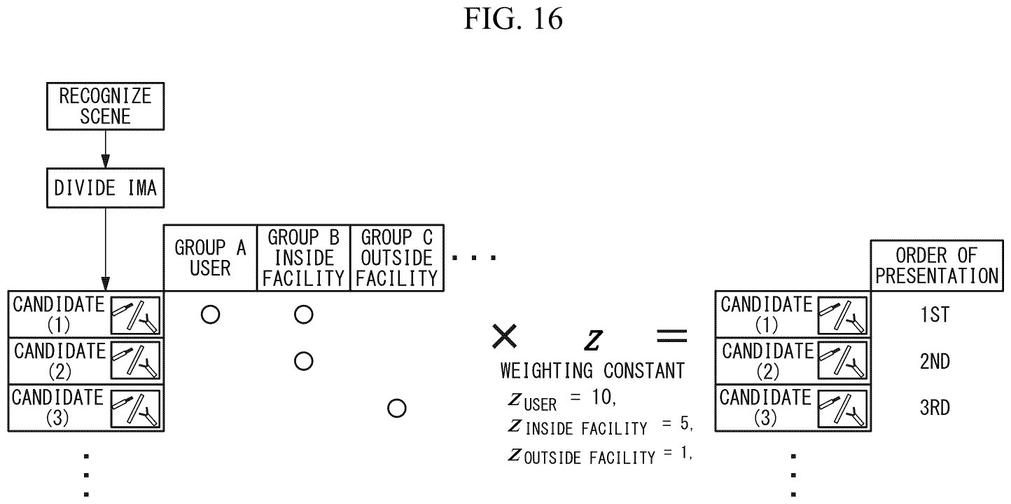

In this case, for example, as shown in FIG. 16, an order of presentation of the library candidates may be set by multiplying tag information associated with each library candidate designated by the user by a weighting constant Z set by the user according to preference.

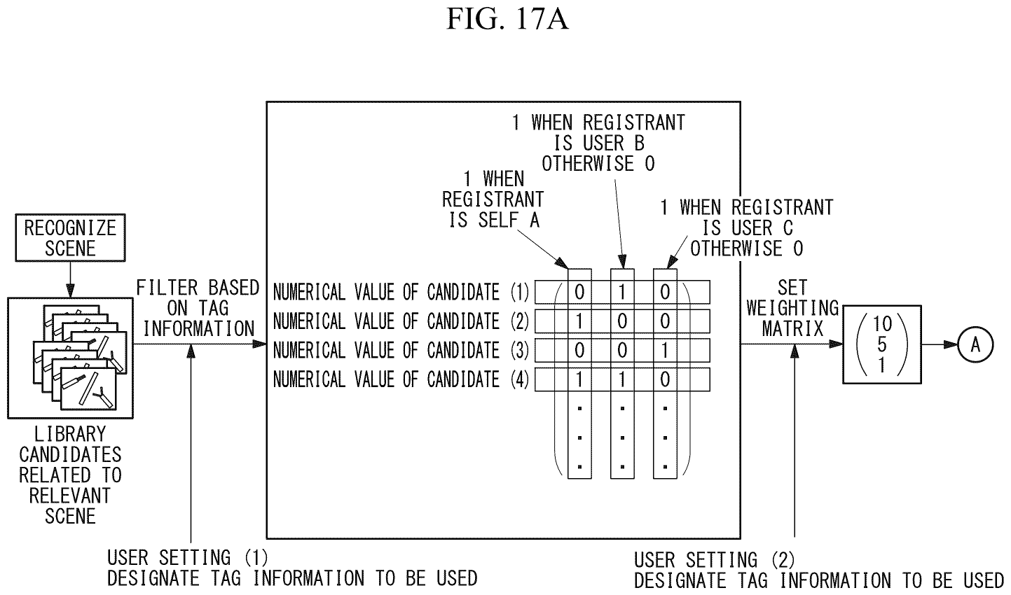

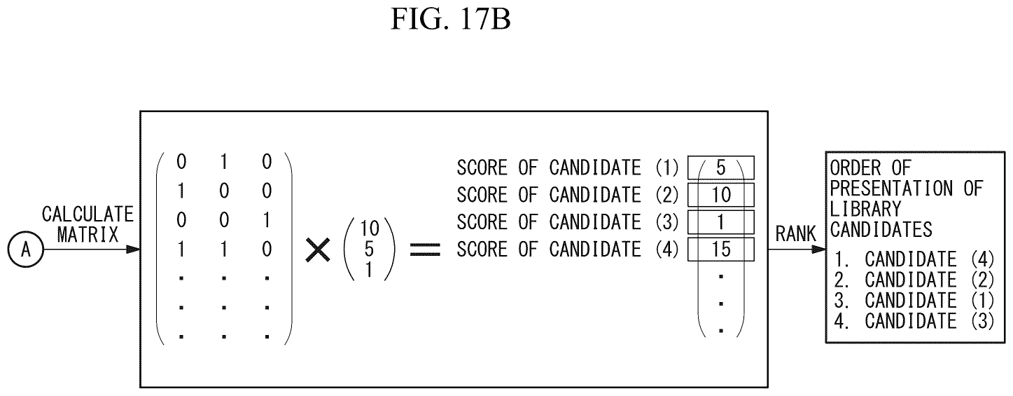

First, the user designates tag information to be used in filtering. In the example shown in FIG. 17A, tag information designated by the user include tag information indicating a library candidate of which a registrant is self A, tag information indicating a library candidate of which a registrant is user B or C other than self A, tag information indicating a library candidate of which an endoscopic image shows no hemorrhage, and tag information indicating a library candidate with a recent registration date.

In addition, the user designates a weighting constant to be used to set a weighting matrix. In the example shown in FIG. 17A, weighting constants designated by the user include 10 for library data of which a registrant is self A, 5 for library data of which a registrant is user B other than self, and 1 for library data of which a registrant is user C other than self.

As shown in FIG. 17A, after extracting a plurality of library candidates associated with the present procedural scene, the determination unit 23 converts the library candidates into a matrix by performing filter processing based on the tag information designated by the user with respect to the plurality of extracted library candidates. In doing so, library candidate with an endoscopic image showing a hemorrhage may be excluded.

FIG. 17A illustrates, with respect to library candidates 1 to n, an n-row, 3-column matrix that represents a registrant of each library candidate as one of self A, a user B, and a user C using a component 1 or a component 0. There may be a plurality of registrants as in a case of a library candidate 4 registered in the course of a surgery during which both self A and the user B had been present.

In addition, the determination unit 23 sets a weighting matrix based on the weighting constant set by the user. FIG. 17A illustrates a 3-row, 1-column matrix made up of a weighting constant 10 of the library candidate of which a registrant is self A, a weighting constant 5 of the library candidate of which a registrant is the user B, and a weighting constant 1 of the library candidate of which a registrant is the user C.

As shown in FIG. 17B, the determination unit 23 calculates a score for each library candidate by multiplying the two matrices. In addition, the determination unit 23 sets an order of priority of the library candidates by assigning, to each library candidate, an order of priority that prioritizes the library candidates in a descending order of scores. In a case where the scores are the same, prioritization may be performed in a reverse chronological order of registration dates.

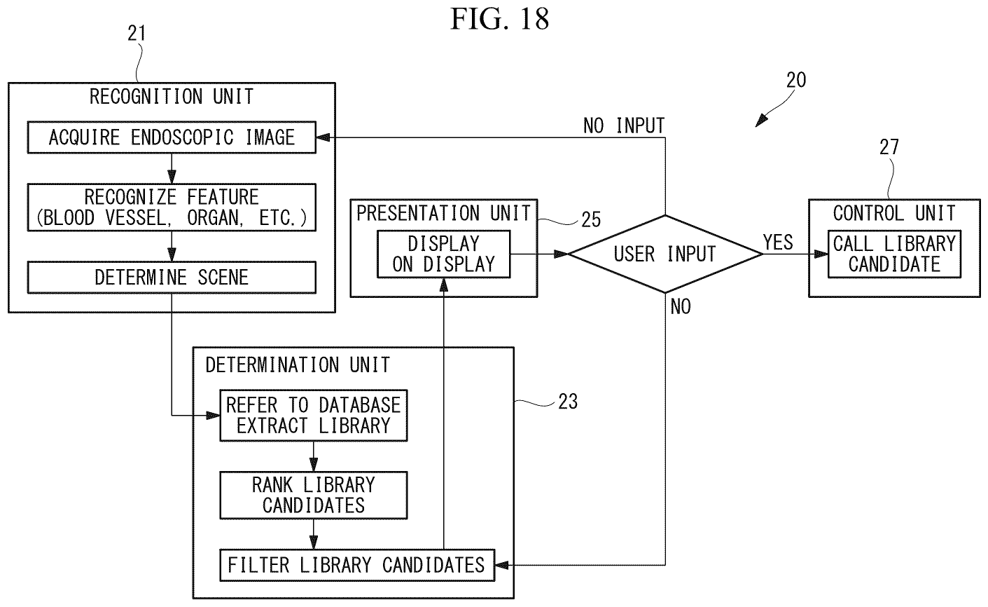

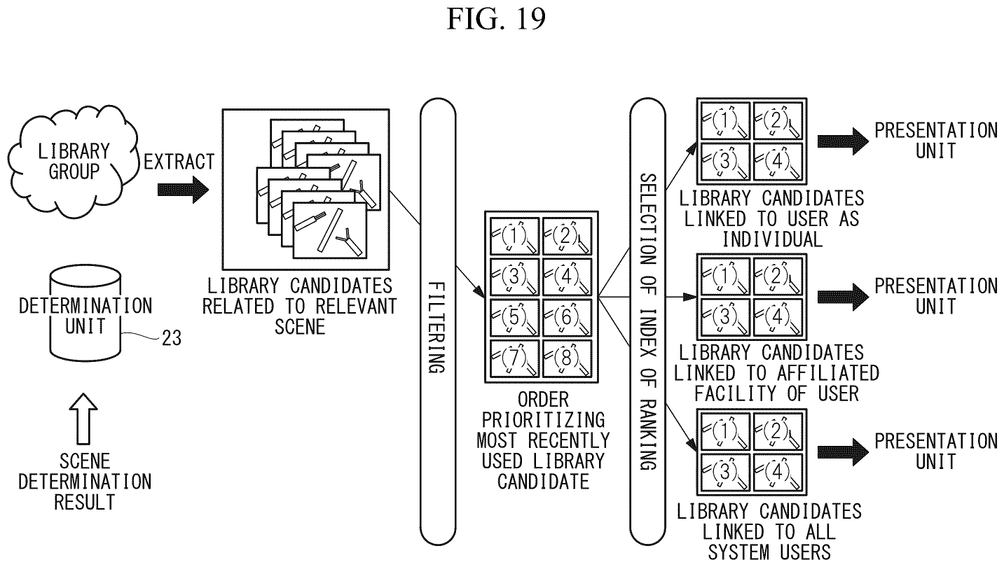

While ranking is performed after filtering in the present modification, an order of the processing steps may be reversed. For example, as shown in FIG. 18 and FIG. 19, after prioritizing library candidates to be presented as an execution candidate according to an order of priority designated by the user with respect to a plurality of extracted library candidates, the determination unit 23 may narrow down library candidates to be selected as an execution candidate according to a refinement condition designated by the user from the prioritized library candidates.

As in the present modification, performing prioritization using a weighting constant set by the user enables prioritization that reflects more detailed demands of the user to be performed.

### Fourth Embodiment

Next, the field-of-view control apparatus 7, the field-of-view control system 1, a field-of-view control method, and a field-of-view control program according to a fourth embodiment of the present disclosure will be described.

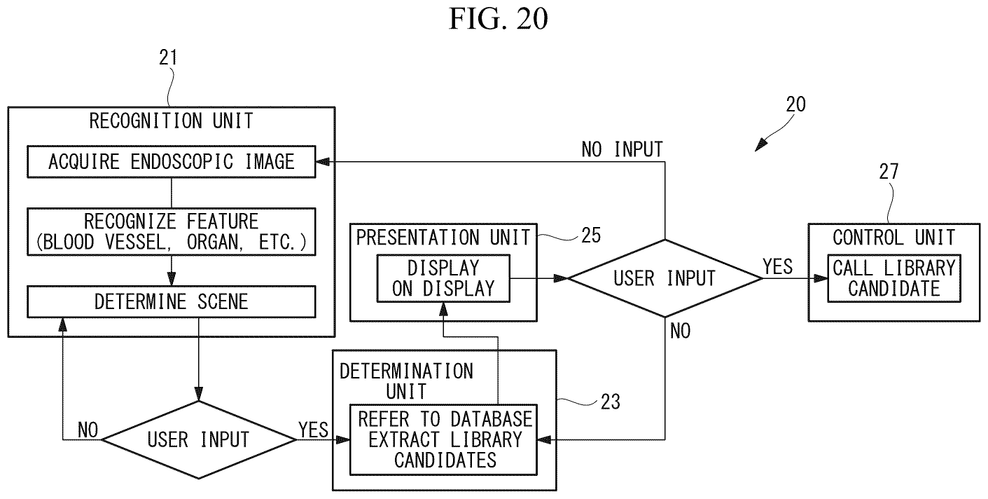

The field-of-view control apparatus 7, the field-of-view control system 1, the field-of-view control method, and the field-of-view control program according to the present embodiment differ from the first embodiment in that, as shown in FIG. 20, the user is made to determine whether or not the present procedural scene determined by the processor 20 is correct.

Hereinafter, portions that share common configurations with the field-of-view control apparatus 7, the field-of-view control system 1, the field-of-view control method, and the field-of-view control program according to the first embodiment will be assigned the same reference signs and descriptions thereof will be omitted.

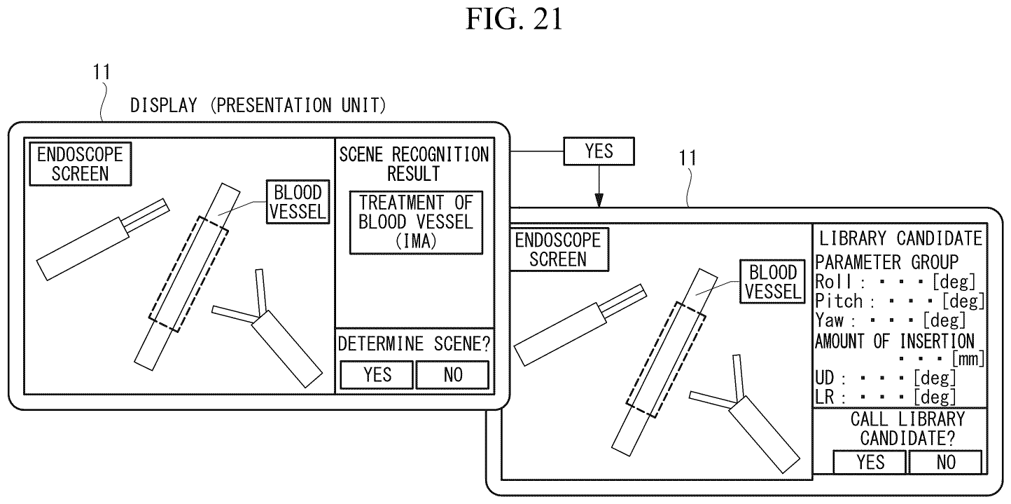

The recognition unit 21 executes scene presentation processing of presenting the user with the determined present procedural scene. Specifically, as shown in FIG. 21, the recognition unit 21 displays information on the determined present procedural scene on the display 11 as a scene recognition result. In addition, the recognition unit 21 accepts an input from the user with respect to whether or not the determination regarding the present procedural scene is correct. In the example shown in FIG. 21, YES is inputted by the user when the determination regarding the present procedural scene by the recognition unit 21 is correct but NO is inputted by the user when the determination is not correct.

When an input to the effect that the information on the present procedural scene displayed on the display 11 matches an actual procedural scene or, in other words, an input to the effect that the information is correct is made by the user, the determination unit 23 executes candidate presentation processing based on the procedural scene. On the other hand, when an input to the effect that the information on the present procedural scene displayed on the display 11 does not match the actual procedural scene or, in other words, an input to the effect that the information is not correct is made by the user, the recognition unit 21 executes re-determination processing of re-determining the present procedural scene.

The recognition unit 21 may execute the re-determination processing based on whatever information that has not been used among the endoscopic image inputted from the electric scope 3, the position and the attitude of the electric scope 3, and the input information of the procedural scene by the user or execute the re-determination processing by appropriately adding whatever information that has not been used.

According to the field-of-view control apparatus 7, the field-of-view control system 1, the field-of-view control method, and the field-of-view control program according to the present embodiment, adding the user's determination to whether or not a determination of the procedural scene derived by the processor 20 is correct enables accuracy of extraction of library data desired by the user to be improved. In addition, since the user can tell at a glance whether a determination of a procedural scene is correct or not, subsequent extractions of library candidates can be carried out in a more efficiently manner based on a correct determination of the procedural scene.

Each of the embodiments described above can be modified as follows.

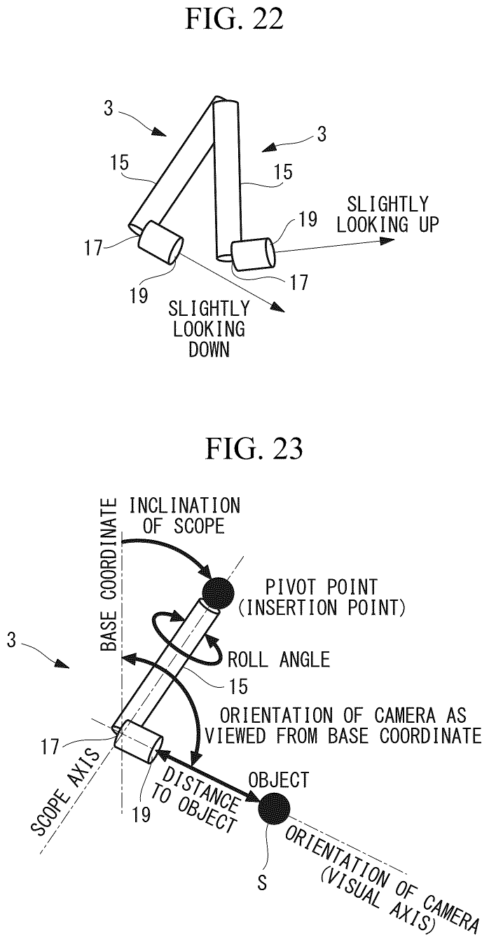

In each of the embodiments described above, amount-of-curvature information representing an amount of curvature of the bending mechanism 17 of the electric scope 3 has been exemplified and described as a relative parameter. For example, as shown in FIG. 22, even when the amount of curvature of the bending mechanism 17 is the same, a degree by which the camera 19 of the electric scope 3 looks up to or looks down to the observation object S changes depending on an inclination of the electric scope 3.

In consideration thereof, for example, as shown in FIG. 23, information indicating an orientation of the camera 19 of the electric scope 3 as viewed from a base coordinate with a center of a base of the robot arm 5 as an origin or, in other words, an orientation of the visual axis of the camera 19 based on the base coordinate may be adopted as the relative parameter in place of the amount-of-curvature information. Hereinafter, this information will be referred to as base coordinate-view orientation information.

The orientation of the camera 19 of the electric scope 3 as viewed from the base coordinate is determined by a sum of an inclination of the electric scope 3 with respect to the base coordinate and an amount of curvature of the bending mechanism 17. By matching the orientation of the camera 19 as viewed from the base coordinate in a given procedural scene with the base coordinate-view orientation information associated with the procedural scene, even if the inclination of the electric scope 3 changes according to a change in a position of biological tissue due to individual variability or the like, the orientation of the camera 19 can be prevented from changing. Therefore, an endoscopic field of view at the time of setting and registration can be more readily and more accurately reproduced.

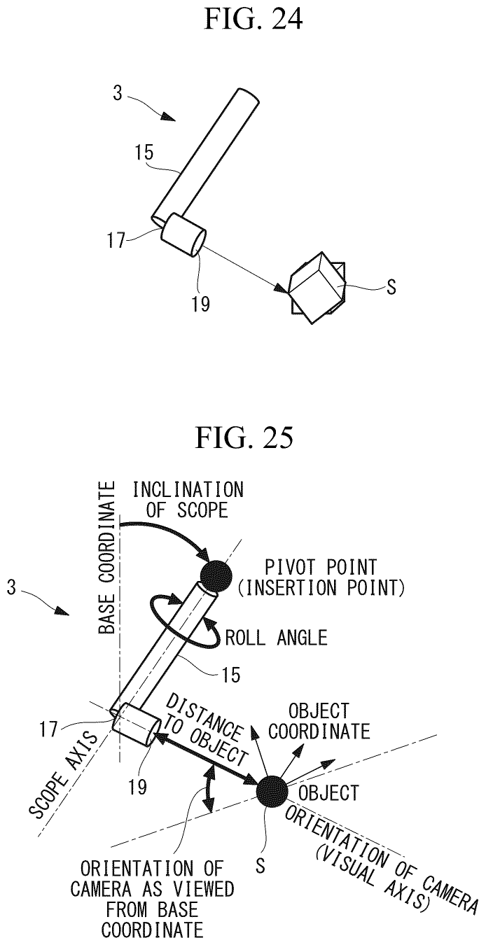

In addition, in a case where amount-of-curvature information of the bending mechanism 17 of the electric scope 3 and orientation information of the visual axis of the camera 19 are adopted as relative parameters, for example, as shown in FIG. 24, when an attitude of the observation object S changes due to an operation of forceps by an assistant or the like, an angle at which the camera 19 looks into the observation object S may relatively change.

In consideration thereof, for example, as shown in FIG. 25, information indicating an orientation of the camera 19 of the electric scope 3 with respect to the observation object S or, in other words, an orientation of the visual axis of the camera 19 as viewed from a coordinate of the observation object S may be adopted as the relative parameter. Hereinafter, this information will be referred to as object-view orientation information. By matching the orientation of the camera 19 of the electric scope 3 as viewed from the observation object S in a given procedural scene to the object-view orientation information associated with the procedural scene, the orientation of the camera 19 of the electric scope 3 is determined in accordance with an attitude of an organ or biological tissue. Therefore, even if an attitude or an orientation of the biological tissue changes due to an operation of forceps by an assistant or the like, a same endoscopic field of view or, in other words, a same angle at which the biological tissue is looked into as during setting and registration can be created.

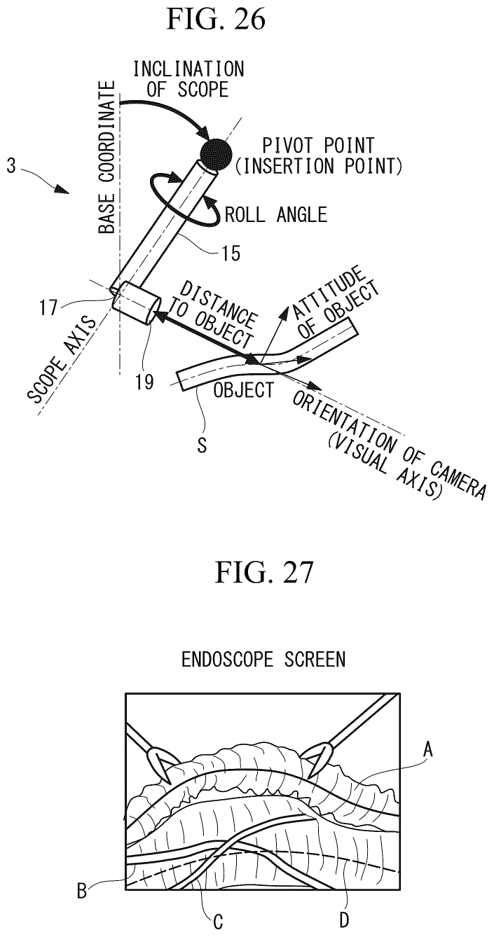

In addition, when scope-shaft roll angle information is adopted as a relative parameter, for example, as shown in FIG. 26, an inclination around the optical axis of the camera 19 of the electric scope 3 of a direction in which an elongated observation object S such as an intestine or a blood vessel runs may be recognized. Furthermore, a parameter that orients the direction in which the intestine or the blood vessel runs in a horizontal direction or a vertical direction of a screen of the display 11 may be set as roll angle information.

In an actual procedure, for example, as shown in FIG. 27, a field of view is adjusted in order to orient the direction in which the intestine or the blood vessel runs or, in other words, a direction in which a dissection line extends in the horizontal direction or the vertical direction of the screen of the display 11. Due to control based on library data, by orienting the direction in which the intestine or the blood vessel runs in the horizontal direction or the vertical direction of the screen of the display 11, an endoscopic field of view that better accommodates individual variability among patients can be obtained. In FIG. 27, reference sign A denotes an intestine, reference sign B denotes a nerve fascicle, reference sign C denotes an IMA (Inferior Mesenteric Artery), and reference sign D denotes a dissection line.

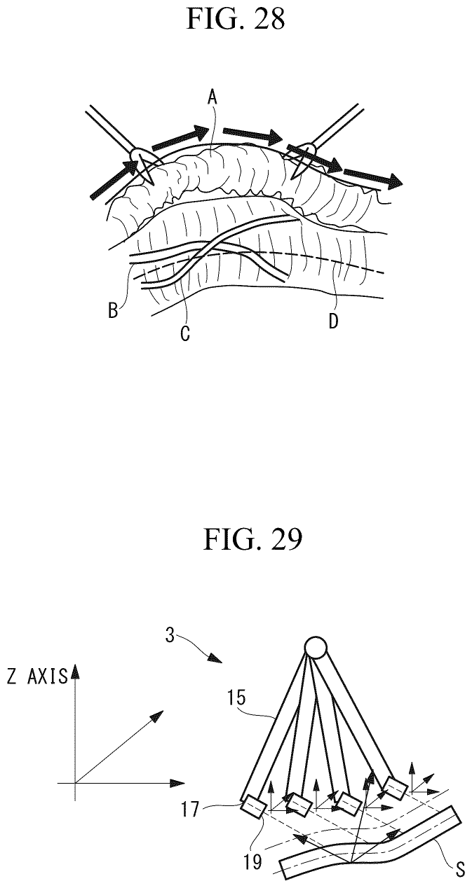

In this case, for example, as shown in FIG. 28, after approximating the direction in which the elongated observation object S such as an intestine or a blood vessel runs with a curve, gradients at a plurality of points are calculated and an average angle of the gradients is calculated. In addition, the direction in which the elongated observation object S runs may be matched with the average angle by adjusting each of the roll angle of the electric scope 3 and the angle of curvatures in the UD (up down) direction and the LR (left right) direction of the bending mechanism 17.

Furthermore, by inputting or detecting a coordinate of the patient or the operating table in advance, for example, as shown in FIG. 29, the roll angle of the electric scope 3 may be matched with the coordinate of the patient or the operating table in a Z-axis direction (usually, vertically upward).

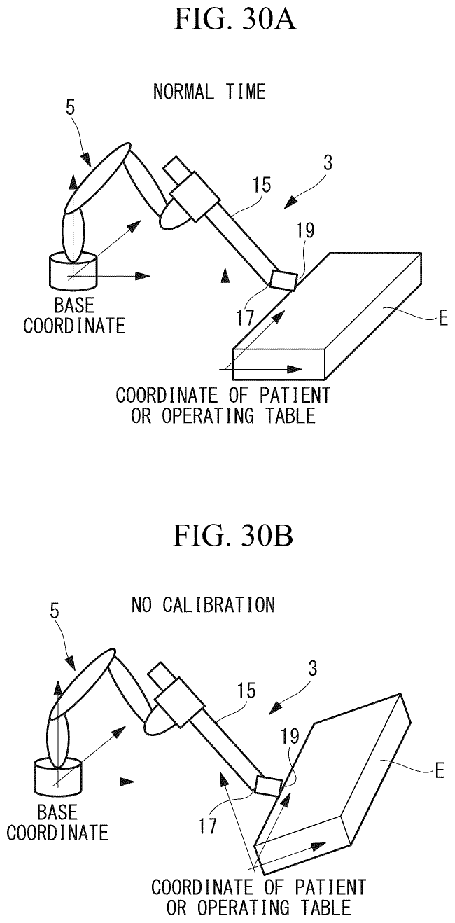

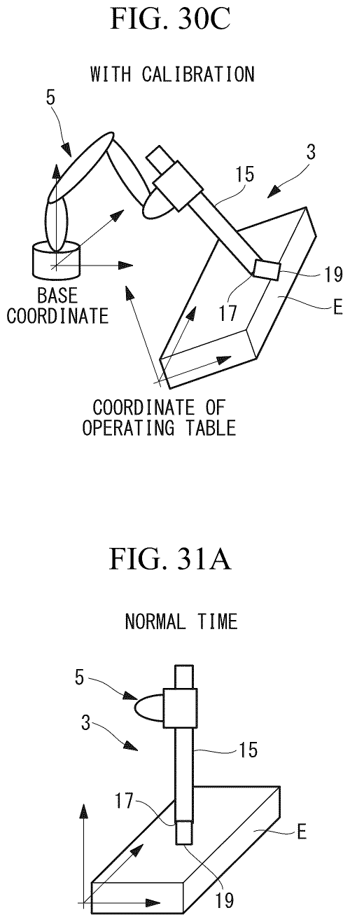

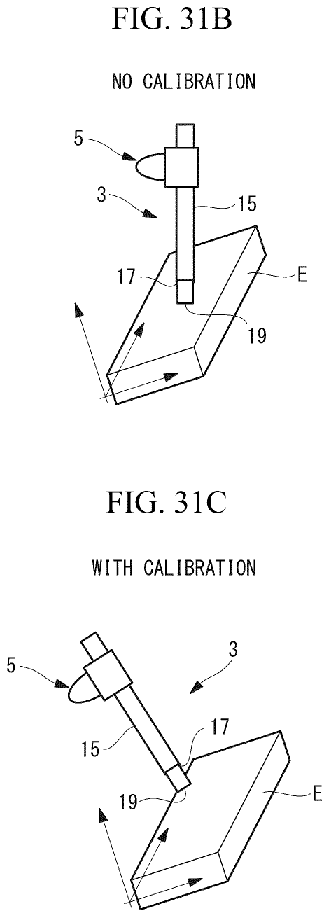

In addition, in each of the embodiments described above, library data or the base coordinate may be calibrated in accordance with an inclination of the patient himself/herself or an inclination of the operating table on which the patient lies. For example, as shown in FIGS. 30A to 30C, after detecting an inclination of the patient or the operating table, the base coordinate may be corrected by adding the detected inclination to the base coordinate. Furthermore, for example, as shown in FIGS. 31A to 31C, after detecting an inclination of the patient or the operating table, library data may be corrected by adding the detected inclination to the library data. In FIGS. 30A to 30C and FIGS. 31A to 31C, reference sign E denotes an operating table or a patient.

In addition, while the scope-shaft roll angle has been exemplified and described as a relative parameter in the present embodiment, for example, a roll angle around the optical axis of the camera 19 of the electric scope 3 or, in other words, a roll angle around the visual axis may be adopted instead of the scope-shaft roll angle. In this case, the control unit 27 may calculate Euler angles (roll, pitch, and yaw) based on the angle of each joint of the robot arm 5 using, for example, forward kinematics of an implement of the electric scope 3 in addition to forward kinematics of the robot arm 5.

Furthermore, the present embodiment can also be applied when performing an expansion operation in a state where a downward endoscopic field of view is created by retracting the electric scope 3 to a vicinity of an insertion point to the patient such as a vicinity of a trocar. In this case, for example, amount-of-curvature information indicating that the amount of curvature of the bending mechanism 17 of the electric scope 3 is zero, scope-shaft forward/backward movement amount information indicating that the amount of forward/backward movement of the electric scope 3 is zero, and scope-shaft roll angle information of which the roll angle takes a desired value may be adopted as relative parameters. A state where the amount of curvature of the bending mechanism 17 is zero is a state where the bending mechanism 17 is oriented in the longitudinal axis direction of the insertion portion 15. A state where the amount of forward/backward movement of the electric scope 3 is zero is a state where the electric scope 3 is positioned in the vicinity of the insertion point to the patient.

Due to such control based on library data, after the amount of curvature of the bending mechanism 17 is restored to zero, the electric scope 3 is retracted until the amount of forward/backward movement becomes zero or, in other words, retracted to an extraction limit and the roll angle of the electric scope 3 is adjusted. By retracting the electric scope 3 to the vicinity of the trocar in a state where the bending mechanism 17 extends in a straight line along the longitudinal axis of the insertion portion 15, a completely downward endoscopic field of view can be created. In this case, the control unit 27 may calculate a present amount of forward/backward movement of the electric scope 3 based on the angle of each joint of the robot arm 5 using forward kinematics of the robot arm 5. The calculated present amount of forward/backward movement of the electric scope 3 is stored by the control unit 27. Fifth embodiment

Next, a field-of-view control apparatus, a field-of-view control system, a field-of-view control method, and a field-of-view control program according to a fifth embodiment of the present disclosure will be described.

The field-of-view control system 1 according to the present embodiment differs from the first to fourth embodiments in that, as shown in FIG. 32, the field-of-view control system 1 comprises an oblique-viewing endoscope (endoscope) 41 not equipped with the bending mechanism 17 instead of the electric scope 3.

Hereinafter, portions that share common configurations with the field-of-view control apparatus 7, the field-of-view control system 1, the field-of-view control method, and the field-of-view control program according to the first to fourth embodiments will be assigned the same reference signs and descriptions thereof will be omitted.

The oblique-viewing endoscope 41 comprises an elongated lens barrel unit 43 to be inserted into a body cavity of the patient and the camera (imaging optical system) 19 provided at a tip portion of the lens barrel unit 43. The oblique-viewing endoscope 41 is arranged in a state where the optical axis of the camera 19 is inclined with respect to a longitudinal axis (central axis) of the lens barrel unit 43. The lens barrel unit 43 comprises a tip surface 43a that is inclined with respect to the longitudinal axis of the lens barrel unit 43 and that is orthogonal to the optical axis of the camera 19. In addition, the oblique-viewing endoscope 41 comprises a ranging function. Reference sign 45 denotes a mounting portion to be supported by the robot arm 5.

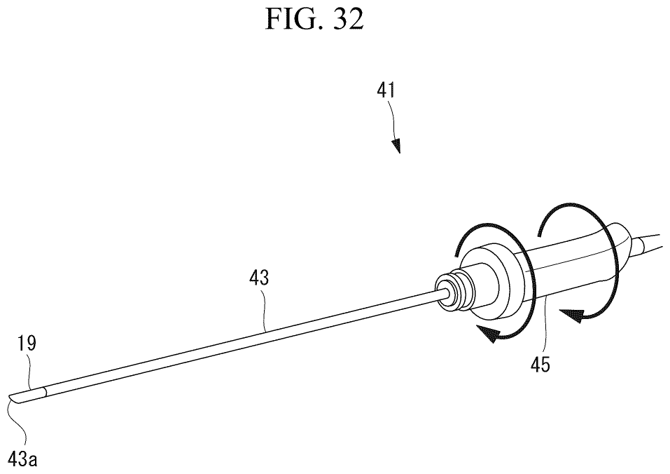

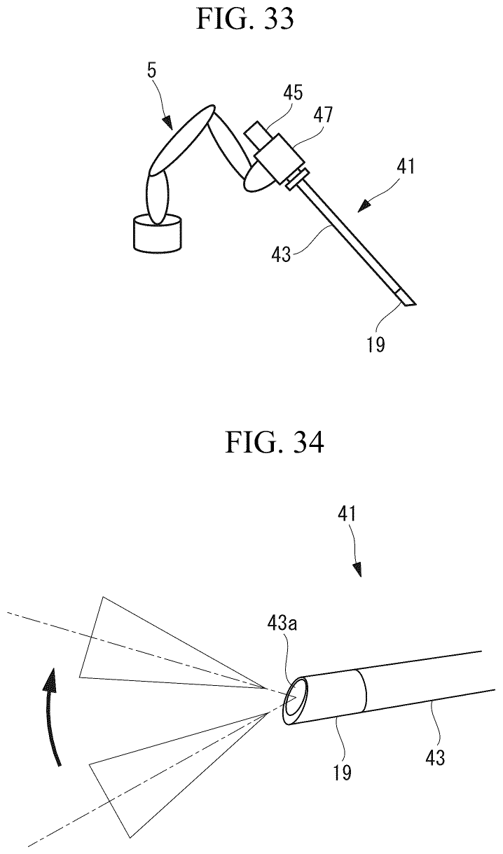

For example, as shown in FIG. 33, the oblique-viewing endoscope 41 is supported by an electric attachment (field-of-view direction changing unit) 47 mounted to a tip portion of the robot arm 5. The electric attachment 47 comprises a lens barrel unit motor that rotates the lens barrel unit 43 around the longitudinal axis and a visual axis motor (both motors not illustrated) that rotates and drives an image rotator (not illustrated) that rotates the visual axis of the camera 19 around an axial line.

Due to the lens barrel unit 43 rotating around the longitudinal axis by being driven by the lens barrel unit motor, as shown in FIG. 34, a direction in which the tip surface 43a at a certain angle with respect to the longitudinal axis of the lens barrel unit 43 is oriented and a direction of the optical axis of the camera 19 or, in other words, a field-of-view direction of the oblique-viewing endoscope 41 can be changed around the longitudinal axis of the lens barrel unit 43.

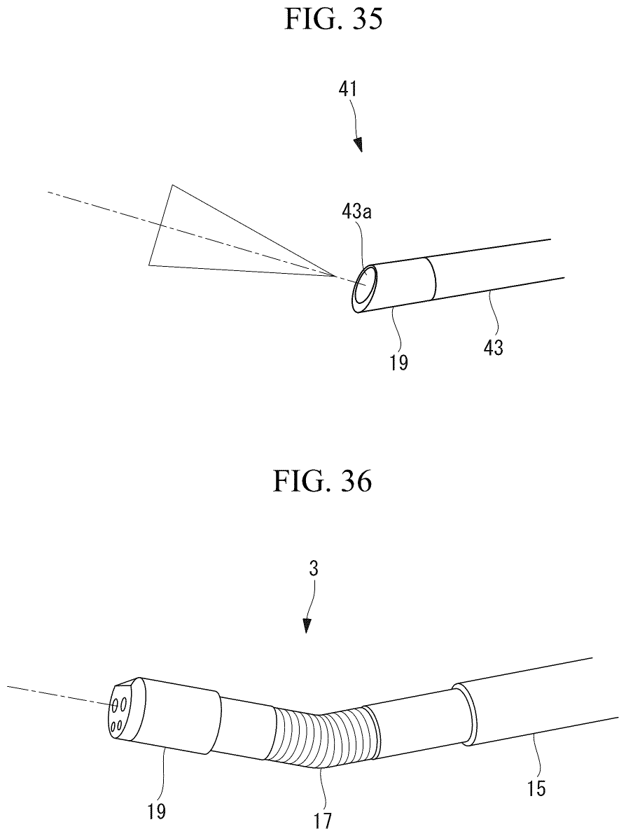

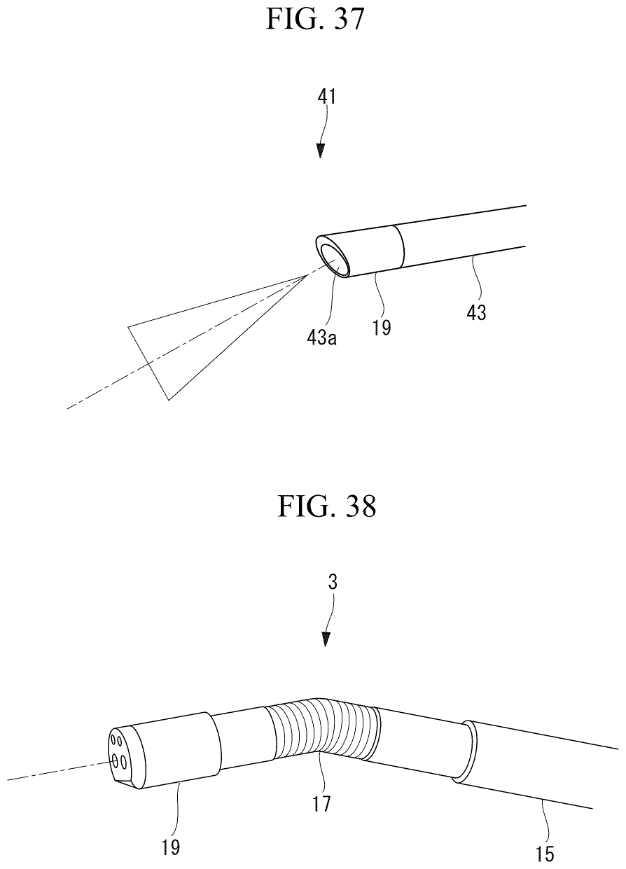

Rotating the lens barrel unit 43 of the oblique-viewing endoscope 41 around the longitudinal axis is equivalent to changing a distribution of upward, downward, leftward, and rightward in a direction of curvature of the bending mechanism 17 while keeping the amount of curvature constant in the electric scope 3. For example, as shown in FIG. 35, a look-up field of view when the tip surface 43a of the oblique-viewing endoscope 41 and the camera 19 are made to face obliquely upward is equivalent to the field of view when the bending mechanism 17 of the electric scope 3 is curved by around 30 degrees in an upward direction as shown in FIG. 36. In addition, for example, as shown in FIG. 37, a look-down field of view when the tip surface 43a of the oblique-viewing endoscope 41 and the camera 19 are made to face obliquely downward is equivalent to the field of view when the bending mechanism 17 of the electric scope 3 is curved by around 30 degrees in a downward direction as shown in FIG. 38.

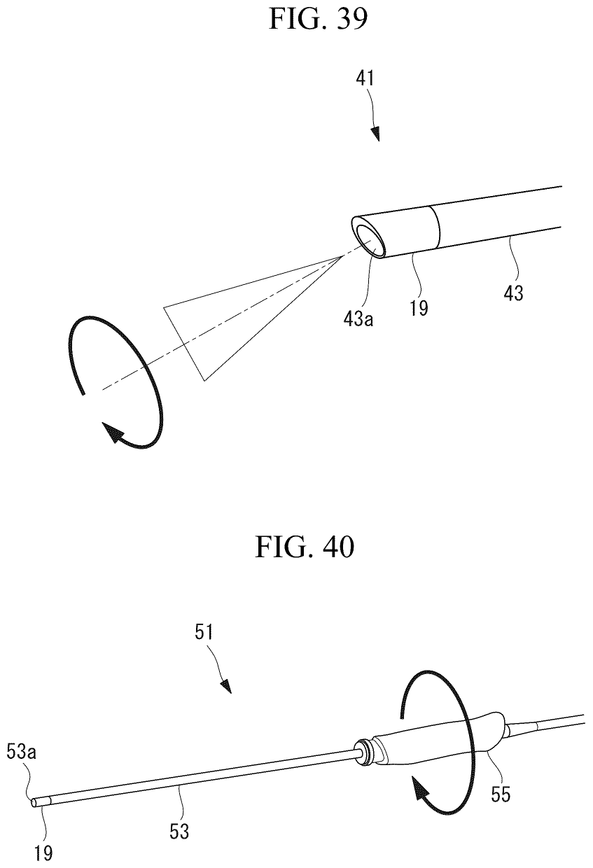

In addition, for example, by rotating the visual axis of the camera 19 around the axial line due to being driven by the visual axis motor as shown in FIG. 39, a vertical direction of an endoscopic image that is acquired by the camera 19 can be rotated around the visual axis.

The plurality of pieces of library data stored in the memory 9 comprises at least one relative parameter related to relative positions and attitudes of the camera 19 of the oblique-viewing endoscope 41 and the observation object S to be photographed by the camera 19. Examples of the relative parameter include distance information of a distance between the camera 19 and the observation object S, rotational angle information of a rotational angle around the longitudinal axis of the lens barrel unit 43, and roll angle information of a roll angle around the visual axis. The roll angle information around the visual axis represents an angle around the axial line of the visual axis of the camera 19. Hereinafter, these pieces of information will be referred to as distance information, lens-barrel-unit angle information, and roll angle-around-visual axis information. In the memory 9, at least any one of distance information, lens-barrel-unit angle information, and roll angle-around-visual axis information for realizing each endoscopic field of view set in advance for each procedural scene is stored in association with each procedural scene.

The field-of-view control method causes at least any one of an angle of the lens barrel unit 43 around the longitudinal axis and a position and an attitude of the oblique-viewing endoscope 41 to be changed based on the relative parameter included in the library data being presented instead of changing at least any one of the angle of the camera 19 and the position and the attitude of the electric scope 3. The field-of-view control program causes processing thereof to be executed by the control unit 27.

For example, when the presented library data includes distance information, the control unit 27 causes an actual distance between the camera 19 and the observation object S that is calculated from image information of the observation object S to match the distance information by controlling at least one of the electric attachment 47 or the robot arm 5.

Specifically, the control unit 27 calculates a trajectory for matching the distance between the camera 19 and the observation object S with the distance information by comparing a present measured distance value to the observation object S and the distance information. Next, based on the calculated trajectory, the control unit 27 uses inverse kinematics of the robot arm 5 to determine a drive amount of each joint required to match the distance between the camera 19 and the observation object S with the distance information. The determined drive amount of each joint is inputted to each motor of the robot arm 5 as an angle command for each joint. In addition, based on the calculated trajectory, the control unit 27 determines a rotational angle around the visual axis and a rotational angle of the lens barrel unit 43 required to match the distance between the camera 19 and the observation object S with the distance information. The determined rotational angle around the visual axis and the determined rotational angle of the lens barrel unit 43 are respectively inputted to the visual axis motor and the lens barrel unit motor of the electric attachment 47 as motor angle commands.

Due to each joint of the robot arm 5 moving according to each angle command and each motor of the electric attachment 47 providing drive according to each motor angle command, an endoscopic field of view in which the distance between the camera 19 and the observation object S matches the distance information is obtained.

In addition, when the presented library data includes lens-barrel-unit angle information, the control unit 27 calculates a trajectory for matching the rotational angle of the lens barrel unit 43 with the lens-barrel-unit angle information by comparing a present angle of the lens barrel unit 43 around the longitudinal axis and the lens-barrel-unit angle information. Furthermore, based on the calculated trajectory, the control unit 27 controls at least one of the electric attachment 47 or the robot arm 5. Accordingly, the angle of the lens barrel unit 43 around the longitudinal axis as calculated from the image information of the observation object S is made to match the lens-barrel-unit angle information. Since control of the electric attachment 47 and the robot arm 5 by the control unit 27 is similar to the case of distance information, a description thereof will be omitted.

Furthermore, when the presented library data includes roll angle information, the control unit 27 calculates a trajectory for matching the angle around the axial line of the visual axis to the roll angle information of the roll angle around the visual axis by comparing a present angle around the axial line of the visual axis of the camera 19 and the roll angle information around the visual axis. In addition, based on the calculated trajectory, the control unit 27 causes the angle around the axial line of the visual axis of the camera 19 that is calculated from image information of the observation object S to match the roll angle information around the visual axis by controlling at least one of the electric attachment 47 or the robot arm 5. Since control of the electric attachment 47 and the robot arm 5 by the control unit 27 is similar to the case of distance information, a description thereof will be omitted.

When the control unit 27 drives the lens barrel unit motor of the electric attachment 47, the control unit 27 calculates a present angular amount around the longitudinal axis of the lens barrel unit 43 by converting a motor angle of the lens barrel unit motor into an angular amount of the lens barrel unit 43. In addition, when the control unit 27 drives the visual axis motor of the electric attachment 47, the control unit 27 calculates a present angular amount around the axial line of the visual axis by converting a motor angle of the visual axis motor into an angular amount around the axial line of the visual axis. The calculated present angular amount of the lens barrel unit 43 and the calculated present angular amount around the axial line of the visual axis are each stored by the control unit 27.

According to the field-of-view control apparatus 7, the field-of-view control system 1, the field-of-view control method, and the field-of-view control program according to the present embodiment, for example, when the angle of the lens barrel unit 43 around the longitudinal axis is changed based on the lens-barrel-unit angle information, the field-of-view direction of the oblique-viewing endoscope 41 is switched to obliquely upward or obliquely downward due to the field-of-view direction of the oblique-viewing endoscope 41 with a certain angle with respect to the longitudinal axis of the lens barrel unit 43 changing around the longitudinal axis of the lens barrel unit 43. Accordingly, the field-of-view direction of the oblique-viewing endoscope 41 can be oriented toward a desired observation object S by simply rotating the lens barrel unit 43 around the longitudinal axis. As a result, even when the oblique-viewing endoscope 41 is adopted as the endoscope, the observation object S desired by the user can be readily placed inside the endoscopic field of view.

### Sixth Embodiment

Next, a field-of-view control apparatus, a field-of-view control system, a field-of-view control method, and a field-of-view control program according to a sixth embodiment of the present disclosure will be described.

The field-of-view control system 1 according to the present embodiment differs from the first to fifth embodiments in that, as shown in FIG. 40, the field-of-view control system 1 comprises a forward-viewing endoscope (endoscope) 51 not equipped with the bending mechanism 17 instead of the electric scope 3 and the oblique-viewing endoscope 41.

Hereinafter, portions that share common configurations with the field-of-view control apparatus 7, the field-of-view control system 1, the field-of-view control method, and the field-of-view control program according to the first to fifth embodiments will be assigned the same reference signs and descriptions thereof will be omitted.

The forward-viewing endoscope 51 comprises an elongated lens barrel unit 53 to be inserted into a body cavity of the patient and the camera (imaging optical system) 19 provided at a tip portion of the lens barrel unit 53. The forward-viewing endoscope 51 is arranged in a state where a longitudinal axis (central axis) of the lens barrel unit 53 coincides with the optical axis of the camera 19. The lens barrel unit 53 comprises a tip surface 53a that is orthogonal to the longitudinal axis of the lens barrel unit 53 and the optical axis of the camera 19. In addition, the forward-viewing endoscope 51 comprises a ranging function. Reference sign 55 denotes a mounting portion to be supported by the robot arm 5.

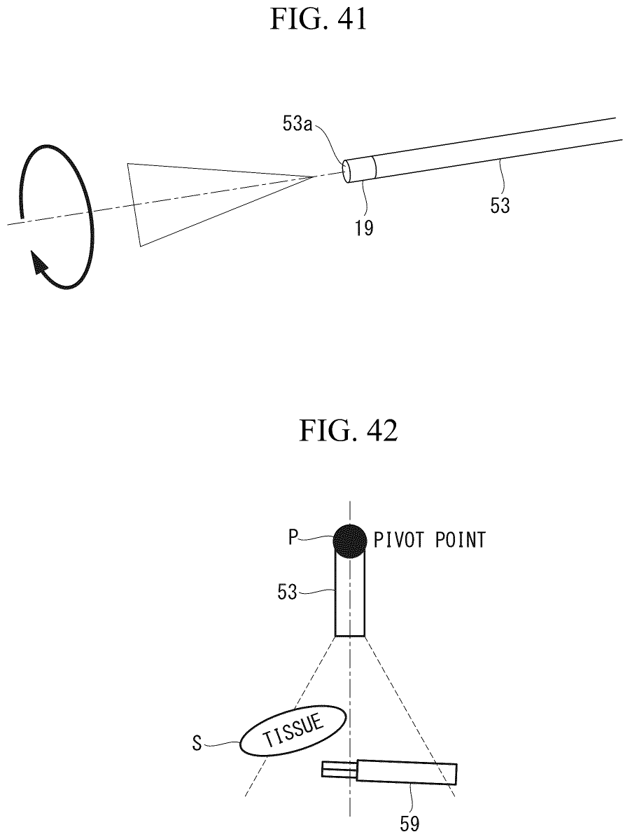

The forward-viewing endoscope 51 is supported by an electric attachment (not illustrated) mounted to a tip portion of the robot arm 5. The electric attachment comprises a visual axis motor that rotates the visual axis of the camera 19 around an axial line or, in other words, around the longitudinal axis of the lens barrel unit 53. By rotating the visual axis of the camera 19 around the axial line as shown in FIG. 41, the forward-viewing endoscope 51 can rotate a vertical direction of an endoscopic image that is acquired by the camera 19 around the visual axis.

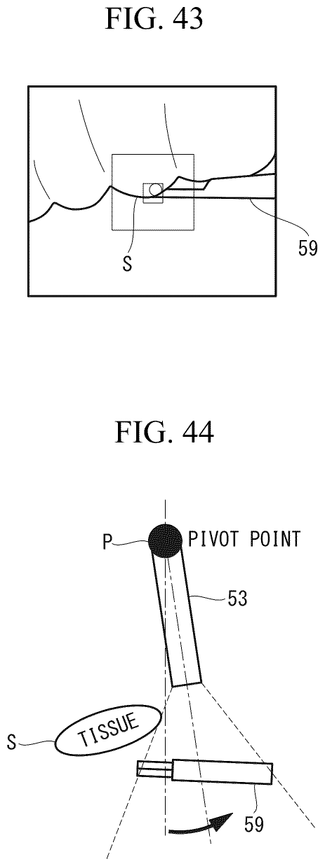

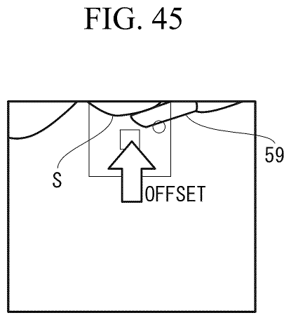

The robot arm 5 functions as a field-of-view direction changing unit that changes an angle around a pivot axis (rotational axis) that is orthogonal to the longitudinal axis of the lens barrel unit 53 in accordance with a change in a position where the observation object S on the screen of the display 11 is captured. For example, when the position where the observation object S in biological tissue or the like is captured is changed to an end in an upper part of the screen of the display 11 from a state where the observation object S is captured at center of the screen, as shown in FIG. 42 and FIG. 44, an angle around a pivot point P that is the mounting portion 55 of the forward-viewing endoscope 51 is changed. Accordingly, as a result of the field-of-view direction of the forward-viewing endoscope 51 being changed to an orientation in which the observation object S is captured at an end of the upper part of the screen, the position of the observation object S on the screen of the display 11 is offset in a direction that intersects with the optical axis of the camera 19 as shown in FIG. 43 and FIG. 45.

When the observation object S is captured at the center of the endoscopic field of view as shown in FIG. 42 and FIG. 43, there is a location that is hidden and hard to view although the observation object S is positioned at the center of the screen. In contrast, when the observation object S is captured at the end of an angle of view as shown in FIG. 44 and FIG. 45, the observation object S is positioned at the end of the screen and the location previously hidden and hard to view becomes visible. In other words, by changing the field-of-view direction of the forward-viewing endoscope 51 to a direction in which the observation object is captured at the end of the angle of view, an endoscopic field of view of an angle of looking into the observation object S can be created. In FIG. 43 and FIG. 45, reference sign 59 denotes a treatment tool and reference sign S denotes the observation object S.

The plurality of pieces of library data stored in the memory 9 comprises at least one relative parameter related to relative positions and attitudes of the camera 19 of the forward-viewing endoscope 51 and the observation object S to be photographed by the camera 19. Examples of the relative parameter include distance information of a distance between the camera 19 and the observation object S, position information on a position where the observation object S is captured on the screen, and roll angle information of a roll angle around the visual axis. Hereinafter, these pieces of information will be referred to as distance information, object position information, and roll angle-around-visual axis information.

In the memory 9, at least any one of distance information, object position information, and roll angle-around-visual axis information for realizing each endoscopic field of view set in advance for each procedural scene is stored in association with each procedural scene. For example, a position where the observation object S on the screen of the display 11 is desirably captured may be set as object position information by an audio operation using a headset 35, a button operation using a hand switch 37, or the like.

The field-of-view control method causes at least any one of an angle of the forward-viewing endoscope 51 around the pivot point P and a position and an attitude of the forward-viewing endoscope 51 to be changed based on the relative parameter included in the presented library data instead of changing at least any one of the angle of the camera 19 and the position and the attitude of the electric scope 3. The field-of-view control program causes processing thereof to be executed by the control unit 27.

For example, when the presented library data includes distance information, the control unit 27 causes an actual distance between the camera 19 and the observation object S that is calculated from image information of the observation object S to match the distance information by controlling at least one of the electric attachment or the robot arm 5.

Specifically, the control unit 27 calculates a trajectory for matching the distance between the camera 19 and the observation object S with the distance information by comparing a present measured distance value to the observation object S and the distance information. Next, based on the calculated trajectory, the control unit 27 uses inverse kinematics of the robot arm 5 to determine a drive amount of each joint required to match the distance between the camera 19 and the observation object S with the distance information. The determined drive amount of each joint is inputted to each motor of the robot arm 5 as an angle command for each joint. In addition, based on the calculated trajectory, the control unit 27 determines a rotational angle around the visual axis required to match the distance between the camera 19 and the observation object S with the distance information. The determined rotational angle around the visual axis is inputted to the visual axis motor of the electric attachment as a motor angle command.

Due to each joint of the robot arm 5 moving according to each angle command and the visual axis motor of the electric attachment rotating according to the motor angle command, an endoscopic field of view in which the distance between the camera 19 and the observation object S matches the distance information is obtained.

In addition, when the presented library data includes object position information, the control unit 27 calculates a trajectory for matching the position of the observation object S on the screen of the display 11 with the object position information by comparing a present position of the observation object S on the screen of the display 11 with the object position information. Furthermore, the control unit 27 controls at least one of the electric attachment or the robot arm 5. Accordingly, the position of the observation object S on the screen as calculated from the image information of the observation object S is made to match the object position information. Since control by the control unit 27 is similar to the case of distance information, a description thereof will be omitted.

Furthermore, when the presented library data includes roll angle information, the control apparatus 7 calculates a trajectory for matching the angle around the axial line of the visual axis to the roll angle information around the visual axis by comparing a present angle of the camera 19 around the axial line of the visual axis and the roll angle information around the visual axis. In addition, based on the calculated trajectory, the control unit 27 causes the angle of the camera 19 around the axial line of the visual axis that is calculated from image information of the observation object S to match the roll angle information around the visual axis by controlling at least one of the electric attachment or the robot arm 5. Since control of the electric attachment and the robot arm 5 by the control unit 27 is similar to the case of distance information, a description thereof will be omitted.

When the control unit 27 drives the visual axis motor of the electric attachment, the control unit 27 calculates a present angular amount around the axial line of the visual axis by converting a motor angle of the visual axis motor into an angular amount around the axial line of the visual axis. The calculated present angular amount is stored by the control unit 27.

While embodiments of the present disclosure have been described in detail with reference to the drawings, specific configurations are not limited to the embodiments and the present disclosure includes design changes and the like made without departing from the scope of the disclosure. For example, the present disclosure is not limited to the disclosure applied to each embodiment and each modification described above and may be applied to embodiments created by appropriately combining the above embodiments and the above modifications without being particularly limited thereto.

In addition, while the field-of-view control system 1 comprises the memory 9 in each embodiment described above, alternatively, the memory 9 may be connected to the field-of-view control apparatus 7 via a communication network such as a LAN, a WAN, or the Internet.

Furthermore, one library candidate extracted by the determination unit 23 is presented in each embodiment described above. Alternatively, a plurality of library candidates extracted by the determination unit 23 may be presented to the user and the user may select a desired library candidate from the plurality of presented library candidates.

In addition, after re-calling a library candidate for which the user had made a selection of not wishing to execute endoscopic field-of-view adjustment, the library candidate may be made selectable.

Furthermore, when a library candidate desired by the user is not presented, the user may create an endoscopic field of view by a manual operation. By registering the created endoscopic field of view as new library data, the endoscopic field of view can be used in a similar procedural scene in subsequent endoscopic procedures.

In addition, for example, while the robot arm 5 that is a 6-axis articulated robot has been exemplified and described as an electrically powered arm in the present embodiment, the electrically powered arm need not have six degrees of freedom and may be a robot arm with fewer degrees of freedom.

As a result, the above-described embodiments lead to the following aspects.

A first aspect of the present disclosure is a field-of-view control apparatus comprising a processor, the processor comprising hardware, the processor being configured to execute: scene determination processing of determining a present procedural scene based on at least one of an endoscopic image of an observation object acquired by an endoscope, a position and an attitude of the endoscope, a field-of-view direction of the endoscope, or input information of the procedural scene by a user; candidate presentation processing of presenting the user with information related to library data for realizing an endoscopic field of view associated with the determined procedural scene as an execution candidate for endoscopic field-of-view adjustment; field-of-view adjustment processing of outputting, in a case where the user wishes to execute the endoscopic field-of-view adjustment based on the presented library data, an instruction to change at least one of the position, the attitude, or the field-of-view direction of the endoscope based on the library data; and candidate change processing of presenting, in a case where the user does not wish to execute the endoscopic field-of-view adjustment based on the presented library data, the user with information related to other library data classified in a same procedural scene as the library data being the execution candidate as another of the execution candidate, wherein the library data includes at least one relative parameter related to relative positions and attitudes of the endoscope and the observation object.

According to the present aspect, in an endoscopic procedure, after the present procedural scene is determined by an actuation of the processor, information related to library data for realizing an endoscopic field of view associated with the procedural scene is presented to the user as an execution candidate for endoscopic field-of-view adjustment.

In addition, when the user wishes to execute endoscopic field-of-view adjustment based on the presented library data, an instruction to change at least one of the position, the attitude, or the field-of-view direction of the endoscope based on the library data is outputted by the processor. Accordingly, a desired endoscopic field of view is provided to the user. On the other hand, when the user does not wish to execute endoscopic field-of-view adjustment based on the presented library data, information related to other library data classified in the same procedural scene as the library data being the execution candidate is presented to the user as another execution candidate. Accordingly, the user can select more preferable library data and an endoscopic field of view desired by the user can be provided.

Therefore, the user can call a desired endoscopic field of view in accordance with a procedural scene by a simple operation of merely selecting, according to the user's preference or a case, library data presented by the processor without having to perform manual adjustment.

In the field-of-view control apparatus according to the aspect described above, the library data may include numerical information representing at least one of the position, the attitude, or the field-of-view direction of the endoscope when the endoscopic field-of-view adjustment is executed based on the library data, and the processor may be configured to present the numerical information to the user as the information related to the library data during the candidate presentation processing and the candidate change processing.

According to this configuration, an endoscopic image desired by the user can be imagined based on information related to library data presented by the processor. In addition, by adopting numerical information as information related to library data, the information can be displayed quicker in proportion to a reduction in an amount of information to be processed as compared to an image.

In addition, in the field-of-view control apparatus according to the aspect described above, the library data may include a schematic view of the endoscopic image representing at least one of the position, the attitude, or the field-of-view direction of the endoscope when the endoscopic field-of-view adjustment is executed based on the library data, and the processor may be configured to present the schematic view of the endoscopic image to the user as the information related to the library data during the candidate presentation processing and the candidate change processing.

By adopting a schematic view of the endoscopic image as information related to library data, features of the field of view can be made more comprehensible than an actual endoscopic image. Accordingly, the field of view after the field-of-view adjustment can be made more imageable.

In addition, in the field-of-view control apparatus according to the aspect described above, the library data may include a schematic view indicating a position and an attitude of the endoscope representing at least one of the position, the attitude, or the field-of-view direction of the endoscope when the endoscopic field-of-view adjustment is executed based on the library data, and the processor may be configured to present the schematic view indicating a position and an attitude of the endoscope to the user as the information related to the library data during the candidate presentation processing and the candidate change processing.

By adopting a schematic view indicating a position and an attitude of the endoscope as information related to library data, a positional relationship between the endoscope and the user during field-of-view adjustment can be made more imageable.

In addition, in the field-of-view control apparatus according to the aspect described above, the library data may include an endoscopic image acquired in a past case representing at least one of the position, the attitude, or the field-of-view direction of the endoscope when the endoscopic field-of-view adjustment is executed based on the library data, and the processor may be configured to present the endoscopic image acquired in a past case to the user as the information related to the library data during the candidate presentation processing and the candidate change processing.

By adopting an endoscopic image acquired in a past case as information related to library data, minute differences in a field of view between pieces of library data can be made more comprehensible.

In the field-of-view control apparatus according to the aspect described above, the library data may be associated with tag information representing an attribute or a feature of the library data in addition to the procedural scene and the processor may be configured to execute filtering processing of narrowing down the library data to be presented as the execution candidate in accordance with a refinement condition based on the tag information designated by the user.

According to this configuration, based on an attribute or a feature of library data, the processor narrows down library data required by the user among a plurality of pieces of library data classified into a same procedural scene. Therefore, library data desired by the user can be called more quickly and accurately.

In the field-of-view control apparatus according to the aspect described above, the library data may be associated with tag information representing an attribute or a feature of the library data in addition to the procedural scene and the processor may be configured to execute ranking processing of presenting, according to an order of priority based on the tag information designated by the user, the library data in a descending order of priority as the execution candidate.

According to this configuration, based on an attribute or a feature of library data, the processor ranks a plurality of pieces of library data classified into a same procedural scene as required by the user. Therefore, library data desired by the user can be called more quickly and efficiently.

In the field-of-view control apparatus according to the aspect described above, the processor may be configured to calculate the order of priority based on the tag information and a weighting constant set by the user.

Using the weighting constant set by the user enables prioritization that reflects more detailed demands of the user to be performed.

In the field-of-view control apparatus according to the aspect described above, the processor may be configured to execute: scene presentation processing of presenting the determined present procedural scene to the user; in a case where the user determines that the presented procedural scene is consistent with an actual procedural scene, the candidate presentation processing based on the procedural scene; and in a case where the user determines that the presented procedural scene is not consistent with the actual procedural scene, re-determination processing of redetermining the present procedural scene.

Adding the user's determination to whether or not a determination of the procedural scene derived by the processor is correct enables accuracy of extraction of library data desired by the user to be improved.

A second aspect of the present disclosure is a field-of-view control system comprising: the field-of-view control apparatus described above; the endoscope; a robot arm configured to change the position and the attitude of the endoscope; and a field-of-view direction changing unit configured to change the field-of-view direction of the endoscope, wherein the processor is configured to control at least one of the robot arm or the field-of-view direction changing unit based on the presented library data.

In the field-of-view control system according to the aspect described above, the field-of-view direction changing unit may comprise a bending mechanism configured to change an angle of an imaging optical system in the endoscope.

According to this configuration, the angle of the imaging optical system changes in accordance with an amount of curvature of the bending mechanism. Accordingly, the field-of-view direction of the endoscope can be changed by a simple configuration of merely curving the bending mechanism.

In the field-of-view control system according to the aspect described above, the endoscope may comprise an oblique-viewing endoscope that has a lens barrel unit housing the imaging optical system and in which an optical axis of the imaging optical system is arranged inclined with respect to a central axis of the lens barrel unit, wherein the field-of-view direction changing unit is configured to change an angle around the central axis of the lens barrel unit.

Changing an angle around the central axis of the lens barrel unit causes a field-of-view direction of the oblique-viewing endoscope with a certain angle with respect to the central axis of the lens barrel unit to change around the central axis of the lens barrel unit and enables the field-of-view direction of the oblique-viewing endoscope to be switched to obliquely upward or obliquely downward. Accordingly, the field-of-view direction of the oblique-viewing endoscope can be oriented toward a desired observation object by simply rotating the lens barrel unit around the central axis. As a result, even when an oblique-viewing endoscope is adopted as the endoscope, an observation object designated by an operator can be readily placed inside the endoscopic field of view.

In the field-of-view control system according to the aspect described above, the endoscope may comprise a forward-viewing endoscope that has a lens barrel unit housing the imaging optical system and in which a central axis of the lens barrel unit and an optical axis of the imaging optical system are arranged consistent with each other, wherein the field-of-view direction changing unit is configured to change an angle around a rotational axis that is orthogonal to the central axis of the lens barrel unit in accordance with a change in a position at which the observation object is captured on a display screen displaying the endoscopic image acquired by the imaging optical system.

Due to an angle around the rotational axis that is orthogonal to the central axis of the lens barrel unit being changed by the field-of-view direction changing unit in accordance with a change in a position at which the observation object is captured on the display screen, the field-of-view direction of the forward-viewing endoscope is changed to a direction which causes the observation object to be captured at a position after the change on the display screen. Accordingly, for example, when the position at which the observation object is captured is changed to an end of the display screen, an endoscopic field of view of an angle of looking into the observation object can be created by changing the field-of-view direction of the forward-viewing endoscope to a direction in which the observation object is captured at the end of an angle of view. As a result, even when a forward-viewing endoscope is adopted as the endoscope, an observation object designated by an operator can be readily placed inside the endoscopic field of view.

The field-of-view control system according to the aspect described above may comprise a memory that stores the library data in association with each procedural scene, wherein the processor may be configured to present, after calling the library data associated with the determined present procedural scene from the memory, information related to the called library data as the execution candidate.

A third aspect of the present disclosure is a field-of-view control method comprising: determining a present procedural scene based on at least one of an endoscopic image of an observation object acquired by an endoscope, a position and an attitude of the endoscope, a field-of-view direction of the endoscope, or input information of the procedural scene by a user; presenting the user with information related to library data for realizing an endoscopic field of view associated with the determined procedural scene as an execution candidate for endoscopic field-of-view adjustment; changing, in a case where the user wishes to execute the endoscopic field-of-view adjustment based on the presented library data, at least one of the position, the attitude, or the field-of-view direction of the endoscope based on the library data; and presenting, in a case where the user does not wish to execute the endoscopic field-of-view adjustment based on the presented library data, the user with information related to other library data classified in a same procedural scene as the library data being the execution candidate as another of the execution candidate, wherein the library data includes at least one relative parameter related to relative positions and attitudes of the endoscope and the observation object.

In the field-of-view control method according to the aspect described above, the library data may include at least one of numerical information, a schematic view of the endoscopic image, a schematic view indicating the position and the attitude of the endoscope, or an endoscopic image acquired in a past case representing at least one of the position, the attitude, or the field-of-view direction of the endoscope when the endoscopic field-of-view adjustment is executed based on the library data, and the at least one of the numerical information, the schematic view of the endoscopic image, the schematic view indicating the position and the attitude of the endoscope, or the endoscopic image acquired in the past case may be presented to the user as the execution candidate and the other of the execution candidate.

In the field-of-view control method according to the aspect described above, the library data may be associated with tag information representing an attribute or a feature of the library data in addition to the procedural scene and the library data to be presented as the execution candidate may be narrowed down in accordance with a refinement condition based on the tag information designated by the user.

A fourth aspect of the present disclosure is a non-transitory computer-readable recording medium storing a field-of-view control program causing a computer to execute: scene determination processing of determining a present procedural scene based on at least one of an endoscopic image of an observation object acquired by an endoscope, a position and an attitude of the endoscope, a field-of-view direction of the endoscope, or input information of the procedural scene by a user; candidate presentation processing of presenting the user with information related to library data for realizing an endoscopic field of view associated with the determined procedural scene as an execution candidate for endoscopic field-of-view adjustment; field-of-view adjustment processing of changing, in a case where the user wishes to execute the endoscopic field-of-view adjustment based on the presented library data, at least one of the position, the attitude, or the field-of-view direction of the endoscope based on the library data; and candidate change processing of presenting, in a case where the user does not wish to execute the endoscopic field-of-view adjustment based on the presented library data, the user with information related to other library data classified in a same procedural scene as the library data being the execution candidate as another of the execution candidate, wherein the library data includes at least one relative parameter related to relative positions and attitudes of the endoscope and the observation object.

In the field-of-view control program according to the aspect described above, the library data may include at least one of numerical information, a schematic view of the endoscopic image, a schematic view indicating the position and the attitude of the endoscope, or an endoscopic image acquired in a past case representing at least one of the position, the attitude, or the field-of-view direction of the endoscope when the endoscopic field-of-view adjustment is executed based on the library data, and the at least one of the numerical information, the schematic view of the endoscopic image, the schematic view indicating the position and the attitude of the endoscope, or the endoscopic image acquired in the past case may be presented to the user as information related to the library data during the candidate presentation processing and the candidate change processing.

In the field-of-view control program according to the aspect described above, the library data may be associated with tag information representing an attribute or a feature of the library data in addition to the procedural scene and the computer may be caused to execute filtering processing of narrowing down library data to be presented as the execution candidate in accordance with a refinement condition based on the tag information designated by the user.

### Example 1

A control apparatus comprising:
one or more processors comprising hardware, the one or more processors being configured to:
   determine a scene based on at least one of an endoscopic image of an observation object acquired by an endoscope, a position and an attitude of the endoscope, a field-of-view direction of the endoscope, or input information by a user;
   output first library data for performing a field of view associated with the determined scene, wherein the first library data includes a parameter related to relative positions and attitudes between the endoscope and the observation object;
   determine whether a user requests to execute field-of-view adjustment based on the library data;
   in response to determining the user requests to execute the field-of-view adjustment, output an instruction to change at least one of the position, the attitude, or the field-of-view direction of the endoscope based on the first library data.

### Example 2

The control apparatus according to Example 1, wherein
the one or more processors is configured to:
   in response to determining the user does not request to execute the field-of-view adjustment, output second information related to second library data classified in the scene.

### Example 3

The control apparatus according to Example 1, wherein
the first library data includes numerical information representing at least one of the position, the attitude, or the field-of-view direction of the endoscope, and
the one or more processors is configured to output the numerical information.

### Example 4

The control apparatus according to Example 1, wherein
the first library data includes a schematic view of the endoscopic image, and
the one or more processors is configured to output the schematic view.

### Example 5

The control apparatus according to Example 1, wherein
the first library data includes a schematic view indicating the position and the attitude of the endoscope, and
the one or more processors is configured to output the schematic view.

### Example 6

The control apparatus according to Example 1, wherein
the first library data includes an endoscopic image acquired in a past case, and
the one or more processors is configured to output the endoscopic image acquired in the past case.

### Example 7

The control apparatus according to Example 1, wherein
the first library data is associated with tag information representing an attribute or a feature of the first library data, and
the one or more processors is configured to filter the first library data in accordance with a refinement condition based on the tag information.

### Example 8

The control apparatus according to Example 1, wherein
the first library data is associated with tag information representing an attribute or a feature of the first library data, and
the one or more processors is configured to output, according to an order of priority based on the tag information, the first library data in a descending order of priority.

### Example 9

The control apparatus according to Example 8, wherein the processor is configured to calculate the order of priority based on the tag information and a weighting constant.

### Example 10

The control apparatus according to Example 1, wherein the one or more processors is configured to:
output the determined scene;
determine whether the user determines that the scene is consistent with an actual procedural scene; and
in response to the determining the user determines that the scene is consistent with the actual procedural scene, output the first information based on the scene.

### Example 11

The control apparatus according to Example 10, wherein the one or more processors is configured to:
in response to the determining the user determines that the scene is not consistent with the actual procedural scene, determine a second scene.

### Example 12

A control system, comprising:
the control apparatus according to Example 1;
the endoscope;
a robot arm configured to change the position and the attitude of the endoscope; and
an actuator configured to change the field-of-view direction of the endoscope,
wherein the one or more processors is configured to control at least one of the robot arm or the actuator based on the first library data.

### Example 13

The control system according to Example 12, wherein the actuator comprises a bending mechanism configured to change an angle of an imaging optical system in the endoscope.

### Example 14

The control system according to Example 12, wherein
the endoscope comprises an oblique-viewing endoscope comprising:
   a lens barrel housing the imaging optical system, wherein an optical axis of the imaging optical system is inclined with respect to a central axis of the lens barrel, and
the actuator is configured to change an angle around the central axis of the lens barrel.

### Example 15

The control system according to Example 12, wherein
the endoscope comprises a forward-viewing endoscope comprising:
   a lens barrel housing the imaging optical system, and wherein a central axis of the lens barrel and an optical axis of the imaging optical system are arranged consistent with each other, and
the actuator is configured to change an angle around a rotational axis that is orthogonal to the central axis of the lens barrel.

### Example 16

The control system according to Example 12, further comprising a memory that stores one or more library data in association with each procedural scene,
wherein the one or more processors is configured to output, after calling the first library data associated with the determined scene from the memory, the first information related to the called library data.

### REFERENCE SIGNS LIST

1 field-of-view control system
3 electric scope (endoscope)
5 robot arm (position/attitude changing unit, field-of-view direction changing unit)
7 field-of-view control apparatus
17 bending mechanism (field-of-view direction changing unit)
20 processor
41 oblique-viewing endoscope (endoscope)
43 lens barrel unit
47 electric attachment (field-of-view direction changing unit)
51 forward-viewing endoscope (endoscope)
53 lens barrel unit
S observation object

1. A field-of-view control apparatus comprising:
one or more processors comprising hardware, the one or more processors being configured to:
scene determination processing of determining a present procedural scene based on at least one of an endoscopic image of an observation object acquired by an endoscope, a position and an attitude of the endoscope, a field-of-view direction of the endoscope, or input information of the procedural scene by a user;
candidate presentation processing of presenting the user with information related to library data for realizing an endoscopic field of view associated with the determined present procedural scene as an execution candidate for endoscopic field-of-view adjustment;
field-of-view adjustment processing of outputting, in a case where the user wishes to execute the endoscopic field-of-view adjustment based on the presented library data, an instruction to change at least one of the position, the attitude, or the field-of-view direction of the endoscope based on the library data; and
candidate change processing of presenting, in a case where the user does not wish to execute the endoscopic field-of-view adjustment based on the presented library data, the user with information related to other library data classified in a same procedural scene as the library data being the execution candidate as another of the execution candidate,
wherein the library data includes at least one relative parameter related to relative positions and attitudes of the endoscope and the observation object.

2. The field-of-view control apparatus according to claim 1, wherein
the library data includes numerical information representing at least one of the position, the attitude, or the field-of-view direction of the endoscope when the endoscopic field-of-view adjustment is executed based on the library data, and
the one or more processors being configured to present the numerical information to the user as the information related to the library data during the candidate presentation processing and the candidate change processing.

3. The field-of-view control apparatus according to claim 1, wherein
the library data includes a schematic view of the endoscopic image representing at least one of the position, the attitude, or the field-of-view direction of the endoscope when the endoscopic field-of-view adjustment is executed based on the library data, and
the one or more processors being configured to present the schematic view of the endoscopic image to the user as the information related to the library data during the candidate presentation processing and the candidate change processing.

4. The field-of-view control apparatus according to claim 1, wherein
the library data includes a schematic view indicating the position and the attitude of the endoscope representing at least one of the position, the attitude, or the field-of-view direction of the endoscope when the endoscopic field-of-view adjustment is executed based on the library data, and the one or more processors being configured to present the schematic view indicating the position and the attitude of the endoscope to the user as the information related to the library data during the candidate presentation processing and the candidate change processing.

5. The field-of-view control apparatus according to claim 1, wherein
the library data includes an endoscopic image acquired in a past case representing at least one of the position, the attitude, or the field-of-view direction of the endoscope when the endoscopic field-of-view adjustment is executed based on the library data, and
the one or more processors being configured to present the endoscopic image acquired in the past case to the user as the information related to the library data during the candidate presentation processing and the candidate change processing.

6. The field-of-view control apparatus according to claim 1, wherein
the library data is associated with tag information representing an attribute or a feature of the library data in addition to the procedural scene, and
the one or more processors being configured to execute filtering processing of narrowing down the library data to be presented as the execution candidate in accordance with a refinement condition based on the tag information designated by the user.

7. The field-of-view control apparatus according to claim 1, wherein
the library data is associated with tag information representing an attribute or a feature of the library data in addition to the procedural scene, and
the one or more processors being configured to executes ranking processing of presenting, according to an order of priority based on the tag information designated by the user, the library data in a descending order of priority as the execution candidate.

8. The field-of-view control apparatus according to claim 7, wherein the processor is configured to calculate the order of priority based on the tag information and a weighting constant set by the user.

9. The field-of-view control apparatus according to claim 1, wherein the one or more processors being further configured executes:
scene presentation processing of presenting the determined present procedural scene to the user;
in a case where the user determines that the presented procedural scene is consistent with an actual procedural scene, the candidate presentation processing is based on the procedural scene; and
in a case where the user determines that the presented procedural scene is not consistent with the actual procedural scene, re-determination processing of redetermining the present procedural scene.

10. A field-of-view control system, comprising:
the field-of-view control apparatus according to claim 1;
the endoscope;
a robot arm configured to change the position and the attitude of the endoscope; and
a field-of-view direction changing unit configured to change the field-of-view direction of the endoscope,
wherein the one or more processors being configured to control at least one of the robot arm or the field-of-view direction changing unit based on the presented library data.

11. The field-of-view control system according to claim 10, wherein the field-of-view direction changing unit comprises a bending mechanism configured to change an angle of an imaging optical system in the endoscope.

12. The field-of-view control system according to claim 10, wherein
the endoscope comprises an oblique-viewing endoscope that has a lens barrel unit housing the imaging optical system and in which an optical axis of the imaging optical system is arranged inclined with respect to a central axis of the lens barrel unit, and
the field-of-view direction changing unit is configured to change an angle around the central axis of the lens barrel unit.

13. The field-of-view control system according to claim 10, wherein
the endoscope comprises a forward-viewing endoscope that has a lens barrel unit housing the imaging optical system and in which a central axis of the lens barrel unit and an optical axis of the imaging optical system are arranged consistent with each other, and
the field-of-view direction changing unit is configured to change an angle around a rotational axis that is orthogonal to the central axis of the lens barrel unit in accordance with a change in a position at which the observation object is captured on a display screen displaying the endoscopic image acquired by the imaging optical system.

14. The field-of-view control system according to claim 10, further comprising a memory that stores the library data in association with each procedural scene,
wherein the one or more processors being configured to present, after calling the library data associated with the determined present procedural scene from the memory, information related to the called library data as the execution candidate.

15. A field-of-view control method comprising:
determining a present procedural scene based on at least one of an endoscopic image of an observation object acquired by an endoscope, a position and an attitude of the endoscope, a field-of-view direction of the endoscope, or input information of the procedural scene by a user;
presenting the user with information related to library data for realizing an endoscopic field of view associated with the determined procedural scene as an execution candidate for endoscopic field-of-view adjustment;
changing, in a case where the user wishes to execute the endoscopic field-of-view adjustment based on the presented library data, at least one of the position, the attitude, or the field-of-view direction of the endoscope based on the library data; and
presenting, in a case where the user does not wish to execute the endoscopic field-of-view adjustment based on the presented library data, the user with information related to other library data classified in a same procedural scene as the library data being the execution candidate as an other of the execution candidate,
wherein the library data includes at least one relative parameter related to relative positions and attitudes of the endoscope and the observation object.

16. The field-of-view control method according to claim 15, wherein
the library data includes at least one of numerical information, a schematic view of the endoscopic image, a schematic view indicating the position and the attitude of the endoscope, or an endoscopic image acquired in a past case representing at least one of the position, the attitude, or the field-of-view direction of the endoscope when the endoscopic field-of-view adjustment is executed based on the library data, and
the at least one of the numerical information, the schematic view of the endoscopic image, the schematic view indicating the position and the attitude of the endoscope, or the endoscopic image acquired in the past case is presented to the user as the execution candidate and the other of the execution candidate.

17. The field-of-view control method according to claim 15, wherein
the library data is associated with tag information representing an attribute or a feature of the library data in addition to the procedural scene, and
the library data to be presented as the execution candidate is narrowed down in accordance with a refinement condition based on the tag information designated by the user.

18. A non-transitory computer-readable recording medium storing a field-of-view control program causing a computer to execute:
scene determination processing of determining a present procedural scene based on at least one of an endoscopic image of an observation object acquired by an endoscope, a position and an attitude of the endoscope, a field-of-view direction of the endoscope, or input information of the procedural scene by a user;
candidate presentation processing of presenting the user with information related to library data for realizing an endoscopic field of view associated with the determined procedural scene as an execution candidate for endoscopic field-of-view adjustment;
field-of-view adjustment processing of changing, in a case where the user wishes to execute the endoscopic field-of-view adjustment based on the presented library data, at least one of the position, the attitude, or the field-of-view direction of the endoscope based on the library data; and
candidate change processing of presenting, in a case where the user does not wish to execute the endoscopic field-of-view adjustment based on the presented library data, the user with information related to other library data classified in a same procedural scene as the library data being the execution candidate as another of the execution candidate,
wherein the library data includes at least one relative parameter related to relative positions and attitudes of the endoscope and the observation object.

19. The non-transitory computer-readable recording medium according to claim 18, wherein
the library data includes at least one of numerical information, a schematic view of the endoscopic image, a schematic view indicating the position and the attitude of the endoscope, or an endoscopic image acquired in a past case representing at least one of the position, the attitude, or the field-of-view direction of the endoscope when the endoscopic field-of-view adjustment is executed based on the library data, and
the at least one of the numerical information, the schematic view of the endoscopic image, the schematic view indicating the position and the attitude of the endoscope, or the endoscopic image acquired in the past case is presented to the user as information related to the library data during the candidate presentation processing and the candidate change processing.

20. The non-transitory computer-readable recording medium according to claim 18, wherein
the library data is associated with tag information representing an attribute or a feature of the library data in addition to the procedural scene, and
the computer is caused to execute filtering processing of narrowing down library data to be presented as the execution candidate in accordance with a refinement condition based on the tag information designated by the user.
